SEQUENCE LISTING (33 sequences)

SEQ ID NO: 1 - DNA, 1539 bp, Artificial Sequence
  Cocal Envelope
atgaacttcctcctgctgacttttatcgtgctgcctctctgctcccacgccaagttctcgattgtgttcccccaatcccaaaaggggaactggaagaatgtgccctcctcgtaccactactgcccgtcctcctccgaccaaaactggcacaacgatctgctcggaatcaccatgaaggtcaagatgcccaagacccataaggctattcaggccgacggctggatgtgccacgccgcgaagtggatcaccacctgtgacttccggtggtacggtccgaagtacatcactcactcgattcactcaattcagccgactagcgagcagtgcaaagagagcatcaagcagacgaagcagggcacatggatgtcccccggattccctccccaaaactgcggatatgcgaccgtgaccgatagcgtggccgtggtggtgcaggccacccctcatcatgtgcttgtggatgagtacaccggagaatggatcgacagccagttcccgaacggaaaatgcgaaaccgaggagtgcgagactgtccacaactccactgtgtggtactccgactacaaggtcacgggcttgtgcgacgcgactttggtggacaccgaaatcaccttctttagcgaggatggaaagaaggagtccatcggcaaaccgaacactggttaccgctccaattacttcgcgtacgaaaagggagacaaagtctgcaagatgaattactgcaagcacgccggtgtcaggctgccatcaggagtgtggttcgaattcgtggaccaggacgtgtacgctgccgcgaagcttccggaatgtccagtcggggcaaccatttccgcaccgactcagacctctgtggatgtgtccctgatcctggacgtcgagagaatcctggactacagcctgtgtcaggagacttggtcgaagattcgctccaagcagcccgtgtcacctgtggatctgtcgtatctggccccgaagaaccctggtaccggcccagcctttaccatcataaacgggaccctgaagtacttcgaaactcggtatattcggattgacatcgacaaccccatcatctcgaaaatggtcggaaagatcagcggatcccagacagaaagggaactctggaccgaatggttcccgtacgagggcgtggaaatcggtccgaacgggatcctgaaaactcctacgggctacaagttccccctcttcatgattgggcatggcatgctggactccgatctccacaagacctcccaagctgaagtgttcgagcaccctcacctggccgaagcacccaagcagctgccagaggaagaaaccctcttcttcggggacaccggaatctcgaagaacccggtggaactgattgagggctggttctcatcatggaagtccaccgtggtcaccttcttcttcgccatcggagtgtttatcctgctttacgtggtggcccgcatcgtgattgccgtgcggtacagataccagggctccaacaacaagcgcatctacaacgatatcgagatgagccggttccgcaagtaa

SEQ ID NO: 2 - protein, 512 aa, Artificial Sequence
  Cocal Envelope
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 3 - DNA, 4286 bp, Artificial Sequence
  Vector
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattctgacactctcaaatcctgcacaacagattcttcatgtttggaccaaatcaacttgtgataccatgctcaaagaggcctcaattatatttgagtttttaatttttatgaaaaaaaaaaaaaaaaacggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 4 - DNA, 5825 bp, Artificial Sequence
  Vector Contaning Cocal Envelope
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattctgacactatgaacttcctcctgctgacttttatcgtgctgcctctctgctcccacgccaagttctcgattgtgttcccccaatcccaaaaggggaactggaagaatgtgccctcctcgtaccactactgcccgtcctcctccgaccaaaactggcacaacgatctgctcggaatcaccatgaaggtcaagatgcccaagacccataaggctattcaggccgacggctggatgtgccacgccgcgaagtggatcaccacctgtgacttccggtggtacggtccgaagtacatcactcactcgattcactcaattcagccgactagcgagcagtgcaaagagagcatcaagcagacgaagcagggcacatggatgtcccccggattccctccccaaaactgcggatatgcgaccgtgaccgatagcgtggccgtggtggtgcaggccacccctcatcatgtgcttgtggatgagtacaccggagaatggatcgacagccagttcccgaacggaaaatgcgaaaccgaggagtgcgagactgtccacaactccactgtgtggtactccgactacaaggtcacgggcttgtgcgacgcgactttggtggacaccgaaatcaccttctttagcgaggatggaaagaaggagtccatcggcaaaccgaacactggttaccgctccaattacttcgcgtacgaaaagggagacaaagtctgcaagatgaattactgcaagcacgccggtgtcaggctgccatcaggagtgtggttcgaattcgtggaccaggacgtgtacgctgccgcgaagcttccggaatgtccagtcggggcaaccatttccgcaccgactcagacctctgtggatgtgtccctgatcctggacgtcgagagaatcctggactacagcctgtgtcaggagacttggtcgaagattcgctccaagcagcccgtgtcacctgtggatctgtcgtatctggccccgaagaaccctggtaccggcccagcctttaccatcataaacgggaccctgaagtacttcgaaactcggtatattcggattgacatcgacaaccccatcatctcgaaaatggtcggaaagatcagcggatcccagacagaaagggaactctggaccgaatggttcccgtacgagggcgtggaaatcggtccgaacgggatcctgaaaactcctacgggctacaagttccccctcttcatgattgggcatggcatgctggactccgatctccacaagacctcccaagctgaagtgttcgagcaccctcacctggccgaagcacccaagcagctgccagaggaagaaaccctcttcttcggggacaccggaatctcgaagaacccggtggaactgattgagggctggttctcatcatggaagtccaccgtggtcaccttcttcttcgccatcggagtgtttatcctgctttacgtggtggcccgcatcgtgattgccgtgcggtacagataccagggctccaacaacaagcgcatctacaacgatatcgagatgagccggttccgcaagtaactcaaatcctgcacaacagattcttcatgtttggaccaaatcaacttgtgataccatgctcaaagaggcctcaattatatttgagtttttaatttttatgaaaaaaaaaaaaaaaaacggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 5 - protein, 5 aa, Artificial Sequence
  Linker
  REPEAT (1)..(5): repeat n times, where n represents an integer of at least 1
GSGGS

SEQ ID NO: 6 - protein, 4 aa, Artificial Sequence
  Linker
  REPEAT (1)..(4): repeat n times, where n represents an integer of at least 1
GGGS

SEQ ID NO: 7 - protein, 5 aa, Artificial Sequence
  Linker
GGGGS

SEQ ID NO: 8 - protein, 4 aa, Artificial Sequence
  Linker
GGSG

SEQ ID NO: 9 - protein, 5 aa, Artificial Sequence
  Linker
GGSGG

SEQ ID NO: 10 - protein, 5 aa, Artificial Sequence
  Linker
GSGSG

SEQ ID NO: 11 - protein, 5 aa, Artificial Sequence
  Linker
GSGGG

SEQ ID NO: 12 - protein, 5 aa, Artificial Sequence
  Linker
GGGSG

SEQ ID NO: 13 - protein, 5 aa, Artificial Sequence
  Linker
GSSSG

SEQ ID NO: 14 - protein, 5 aa, Artificial Sequence
  Linker
GGGGS

SEQ ID NO: 15 - protein, 15 aa, Artificial Sequence
  Linker
GGGGSGGGGSGGGGS

SEQ ID NO: 16 - DNA, 45 bp, Artificial Sequence
  Linker
ggtggcggtggctcgggcggtggtgggtcgggtggcggcggatct

SEQ ID NO: 17 - protein, 5 aa, Artificial Sequence
  Hinge
DKTHT

SEQ ID NO: 18 - protein, 4 aa, Artificial Sequence
  Hinge
CPPC

SEQ ID NO: 19 - protein, 15 aa, Artificial Sequence
  Hinge
CPEPKSCDTPPPCPR

SEQ ID NO: 20 - protein, 12 aa, Artificial Sequence
  Hinge
ELKTPLGDTTHT

SEQ ID NO: 21 - protein, 10 aa, Artificial Sequence
  Hinge
KSCDKTHTCP

SEQ ID NO: 22 - protein, 7 aa, Artificial Sequence
  Hinge
KCCVDCP

SEQ ID NO: 23 - protein, 7 aa, Artificial Sequence
  Hinge
KYGPPCP

SEQ ID NO: 24 - protein, 15 aa, Artificial Sequence
  Hinge
EPKSCDKTHTCPPCP

SEQ ID NO: 25 - protein, 12 aa, Artificial Sequence
  Hinge
ERKCCVECPPCP

SEQ ID NO: 26 - protein, 17 aa, Artificial Sequence
  Hinge
ELKTPLGDTTHTCPRCP

SEQ ID NO: 27 - protein, 12 aa, Artificial Sequence
  Hinge
SPNMVPHAHHAQ

SEQ ID NO: 28 - protein, 15 aa, Artificial Sequence
  Hinge
EPKSCDKTYTCPPCP

SEQ ID NO: 29 - protein, 4 aa, Artificial Sequence
  Furin cleavage site
  misc_feature (2)..(2): Xaa can be any naturally occurring amino acid
RXKR

SEQ ID NO: 30 - protein, 4 aa, Artificial Sequence
  Furin cleavage site
  misc_feature (2)..(2): Xaa can be any naturally occurring amino acid
RXRR

SEQ ID NO: 31 - protein, 5 aa, Artificial Sequence
  Furin cleavage site
  MISC_FEATURE (1)..(1): Xaa is K or R
  misc_feature (3)..(3): Xaa can be any naturally occurring amino acid
  MISC_FEATURE (4)..(4): Xaa is K or R
XRXXR

SEQ ID NO: 32 - protein, 4 aa, Artificial Sequence
  Furin cleavage site
  misc_feature (2)..(3): Xaa can be any naturally occurring amino acid
RXXR

SEQ ID NO: 33 - protein, 4 aa, Artificial Sequence
  Furin cleavage site
RQKR

(19) United States
(12) Patent Application Publication
(10) Pub. No.: US 2023/0323394 A1
(43) Pub. Date: Oct. 12, 2023

(54) IMPROVED GENERATION OF LENTIVIRAL VECTORS FOR T CELL TRANSDUCTION USING COCAL ENVELOPE

(71) Applicant: The Trustees of the University of Pennsylvania, Philadelphia, PA (US)

(72) Inventor: James L. Riley, Downingtown, PA (US)

(21) Appl. No.: 18/043,462

(22) PCT Filed: Aug. 31, 2021
(86) PCT No.: PCT/US2021/048331

§ 371 Date: Feb. 28, 2023

Related U.S. Application Data
(60) Provisional application No. 63/073,194, filed on Sep. 1, 2020.

(51) Int. Cl. C12N 15/86; C12N 5/0783; C07K 14/005; A61K 39/00

(52) U.S. Cl. CPC C12N 15/86; C12N 5/0636; C07K 14/005; A61K 39/4611; A61K 39/4631; C12N 2500/90; C12N 2740/15052; C12N 2740/15043; C12N 2760/20222; C12N 2740/16022; A61K 2239/26; A61K 2239/21; A61K 2239/22; A61K 2239/13

(57) ABSTRACT

The present disclosure provides compositions and methods for delivering a nucleic acid sequence encoding a chimeric antigen receptor (CAR) to an immune cell using a retroviral vector comprising an optimized Cocal vesiculovirus envelope protein.

### CROSS-REFERENCE TO RELATED APPLICATION

The present application is entitled to priority under 35 U.S.C. § 119(e) to U.S. Provisional Patent Application No. 63/073,194 filed Sep. 1, 2020, which is hereby incorporated by reference in its entirety herein.

### BACKGROUND OF THE INVENTION

Retroviral vectors, including lentiviral vectors, provide for gene therapies in preclinical animal models, veterinary medicine, clinical studies, and therapeutic applications. Lentiviral vectors can efficiently transduce quiescent cells compared to gammaretroviral vectors. It is thought that the increased efficiency is at least due to the ability of lentiviruses to enter a nucleus of an infected cell not only during mitosis (e.g. cell division) but also throughout the life cycle of the cell. Lentiviral vectors have the advantage that they do not integrate very close to promoter regions compared to, for example, gammaretroviral vectors. Accordingly, the risk of gene disruption, cancer, and teratoma formation is believed to be lower for lentiviral vectors than for gammaretroviral vectors. Lentiviruses also have the advantage of being designed to be self-inactivating, which also improves the safety profile.

For the purposes of gene therapy, one might either want to limit or expand the range of cells susceptible to transduction by a gene therapy vector. To this end, many vectors have been developed in which the endogenous viral envelope proteins have been replaced by either envelope proteins from other viruses, or by chimeric proteins. Such chimera would consist of those parts of the viral protein necessary for incorporation into the virion as well as sequences meant to interact with specific host cell proteins. Viruses in which the envelope proteins have been replaced as described are referred to as pseudotyped viruses. The most popular lentivirus pseudotype is a Indiana vesiculovirus (also known as “vesicular stomatitis virus” and “vesicular stomatitis Indiana virus”) envelope glycoprotein (VSV-G). While VSV-G has been shown to have broad efficacy and can be produced in high titers, VSV-G is also associated with toxicity and instability in the cell lines used to generate and package the retroviral particle (i.e. “producer cell) comprising or encapsulated by VSV-G. Once administered in vivo, VSV-G can be inactivated by human serum compliment, thus reducing its efficacy and causing an adverse complement-dependent immune response to the VSV-G in the patient. The Cocal vesiculovirus is in the same genus but is serologically distinct from Indiana vesiculovirus.

There is a need in the art for lentiviral vectors with increased titers, improved efficacy, and lower toxicity in the patient. The present invention addresses this need.

### SUMMARY OF THE INVENTION

As described herein, the present invention relates to compositions and methods for delivering a nucleic acid sequence encoding a chimeric antigen receptor (CAR) to an immune cell using a retroviral vector comprising an optimized Cocal vesiculovirus envelope protein.

In one aspect, the invention includes a method for delivering a nucleic acid encoding a chimeric antigen receptor (CAR) to an immune cell or precursor cell thereof. The method comprises introducing into the cell: a) a transfer plasmid comprising a nucleotide sequence encoding a CAR, b) a retroviral vector comprising a nucleotide sequence encoding a Cocal vesiculovirus envelope protein, c) a plasmid comprising a nucleotide sequence encoding a retroviral Rev protein, and d) at least one plasmid comprising a nucleotide sequence encoding a retroviral Gag protein and a retroviral Pol protein. The amount of transfer plasmid introduced is higher than the amount of the retroviral vector comprising a nucleotide sequence encoding a Cocal vesiculovirus envelope protein.

In certain embodiments, the amount of transfer plasmid introduced is at least 2 times (×), 3×, 4×, 5×, 6×, 7×, 8×, 9×, 10×, or 20× the amount of the vector comprising a nucleotide sequence encoding a Cocal vesiculovirus envelope protein.

In certain embodiments, the nucleotide sequence encoding the Cocal vesiculovirus envelope is at least 80%, at least 85%, at least 90%, at least 95%, at least 96%, at least 97%, at least 98%, at least 99%, or 100% identical to SEQ ID NO: 1.

In certain embodiments, the expression of the envelope protein is under control of a transcriptional regulatory element. In certain embodiments, the transcriptional regulatory element is a eukaryotic promoter. In certain embodiments, the transcriptional regulatory element is a constitutive promoter.

In certain embodiments, the Cocal vesiculovirus envelope protein comprises an amino acid sequence at least 80%, at least 85%, at least 90%, at least 95%, at least 96%, at least 97%, at least 98%, at least 99%, or 100% identical to SEQ ID NO: 2.

In certain embodiments, the retroviral vector comprises a nucleotide sequence at least 80%, at least 85%, at least 90%, at least 95%, at least 96%, at least 97%, at least 98%, at least 99%, or 100% identical to SEQ ID NO: 4.

In certain embodiments, the CAR comprises an antigen-binding domain, a transmembrane domain, and an intracellular domain.

In certain embodiments, the antigen-binding domain is selected from the group consisting of a full-length antibody or antigen-binding fragment thereof, a Fab, a single-chain variable fragment (scFv), or a single-domain antibody. In certain embodiments, the antigen-binding domain specifically binds a target antigen selected from the group consisting of CD4, CD19, CD20, CD22, BCMA, CD123, CD133, EGFR, EGFRvIII, mesothelin, Her2, PSMA, CEA, GD2, IL-13Ra2, glypican-3, CIAX, LI-CAM, CA 125, CTAG1B, Mucin 1 (MUC1), TnMUC1, glypican-2 (GPC2), cancer cell-associated GPC2, Glycosyl-phosphatidylinositol (GPI)-linked GDNF family α-receptor 4 (GFRα4; GFRalpha4), and Folate receptor-alpha.

In certain embodiments, the CAR further comprises a hinge region.

In certain embodiments, the transmembrane domain is selected from the group consisting of an artificial hydrophobic sequence, a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), ICOS (CD278), or CD154, and a transmembrane domain derived from a killer immunoglobulin-like receptor (KIR).

In certain embodiments, the intracellular domain comprises a costimulatory signaling domain and an intracellular signaling domain. In certain embodiments, the intracellular domain comprises a costimulatory domain of a protein selected from the group consisting of a TNFR superfamily protein, CD27, CD28, 4-1BB (CD137), OX40 (CD134), PD-1, CD7, LIGHT, CD83L, DAP10, DAP12, CD27, CD2, CDS, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR), or a variant thereof. In certain embodiments, the intracellular signaling domain comprises an intracellular domain selected from the group consisting of cytoplasmic signaling domains of a human CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In certain embodiments, the immune cell is a T cell, a natural killer cell, a cytotoxic T lymphocyte, or a regulatory T cell. In certain embodiments, the T cell is a CD8+ T cell. In certain embodiments, the T cell is a CD4+ T cell. In certain embodiments, the T cell is a regulatory T cell.

In certain embodiments, the retroviral vector is selected from the group consisting of a lentiviral vector, an alpharetroviral, a betaretroviral, a gammaretroviral, a deltaretrovirus, and an epsilonretrovirus.

In certain embodiments, the Cocal vesiculovirus envelope protein is human codon-optimized.

In certain embodiments, the method is scaled-up. In certain embodiments, the method further comprises adapting the cells for growth in suspension. In certain embodiments, the method further comprises adapting the cells to grow in serum-free cultures.

In another aspect, the invention includes a composition comprising an immune cell or precursor cell thereof comprising a CAR, wherein the cell is produced by any of the methods contemplated herein. In certain embodiments, the composition is GMP compliant.

In another aspect, the invention includes a method for delivering a nucleic acid sequence encoding a chimeric antigen receptor (CAR) to an immune cell or precursor cell thereof. The method comprises transducing the cell with a Cocal vesiculovirus envelope pseudotyped retroviral particle generated in a host cell, wherein the Cocal vesiculovirus envelope pseudotyped retroviral particle comprises: a transfer plasmid comprising a nucleotide sequence encoding a CAR, a retroviral vector comprising a nucleotide sequence encoding a Cocal vesiculovirus envelope protein, a plasmid comprising a nucleotide sequence encoding a retroviral Rev protein, and at least one plasmid comprising a nucleotide sequence encoding a retroviral Gag protein and a retroviral Pol protein.

In another aspect, the invention includes a method for delivering a nucleic acid sequence encoding a chimeric antigen receptor (CAR) to an immune cell. The method comprises introducing into a host cell a transfer plasmid comprising a nucleotide sequence encoding a CAR, a retroviral vector comprising a nucleotide sequence encoding a Cocal vesiculovirus envelope protein, a plasmid comprising a nucleotide sequence encoding a retroviral Rev protein, and at least one plasmid comprising a nucleotide sequence encoding a retroviral Gag protein and a retroviral Pol protein, wherein the host cell produces a Cocal vesiculovirus envelope pseudotyped retroviral particle. The method further comprises harvesting the Cocal vesiculovirus envelope pseudotyped retroviral particle; and transducing the immune cell with the Cocal vesiculovirus envelope pseudotyped retroviral vector particle, wherein the transduced immune cell expresses the CAR encoded by the nucleotide sequence of the transfer plasmid.

In certain embodiments, the amount of transfer plasmid introduced into the host cell is higher than the amount of the retroviral vector comprising a nucleotide sequence encoding a Cocal vesiculovirus envelope protein.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 illustrates an exemplary vector encoding the codon optimized Cocal vesiculovirus envelope glycoprotein (hereinafter “Cocal-G”).

FIG. 2 illustrates the nucleotide sequence of the vector of FIG. 1, including the codon optimized Cocal vesiculovirus envelope glycoprotein.

FIG. 3 illustrates an exemplary amino acid sequence of the codon optimized Cocal vesiculovirus envelope glycoprotein.

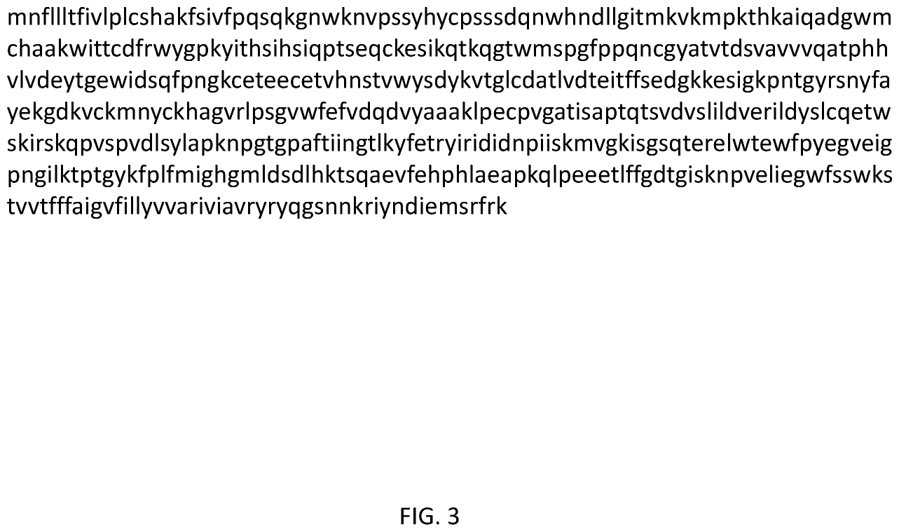

FIG. 4 illustrates the finding that codon-optimized Cocal-G enveloped lentiviral particles (Cocal-G ENV) exhibit better transduction efficiencies in primary human T cells than that of vesicular stomatitis virus (Indiana vesiculovirus) (VSV-G) enveloped lentiviral particles, especially after adjusting the envelope and transfer plasmid concentrations.

FIG. 5 depicts flow cytometry data from FIG. 4 graphed as the percentage of cells expressing GFP and the total mean fluorescence intensity (MFI) of the cells.

FIG. 6 illustrates the finding that Cocal-G ENV enhances lentivirus transduction efficiency in CD8+ T cells, especially after adjusting the envelope and transfer plasmid concentrations.

FIG. 7 illustrates the finding that Cocal-G enhances the transduction efficiency of CD4 CARs in CD8 T cells.

### DETAILED DESCRIPTION

The present invention provides compositions and methods for delivering a nucleic acid sequence encoding a chimeric antigen receptor (CAR) to an immune cell using a retroviral vector comprising an optimized Cocal vesiculovirus envelope protein. In certain embodiments, the method comprises introducing into an immune cell a transfer plasmid comprising a nucleotide sequence encoding a CAR, a retroviral vector comprising a nucleotide sequence encoding a Cocal vesiculovirus envelope protein, a plasmid comprising a nucleotide sequence encoding a retroviral Rev protein, and at least one plasmid comprising a nucleotide sequence encoding a retroviral Gag protein and a retroviral Pol protein, wherein the amount of transfer plasmid introduced is higher than the amount of the retroviral vector comprising a nucleotide sequence encoding a Cocal vesiculovirus envelope protein.

In certain embodiments, the invention provides a vector for the expression of a Cocal vesiculovirus envelope glycoprotein, a viral particle comprising a Cocal vesiculovirus envelope glycoprotein, a nucleic acid encoding a Cocal vesiculovirus envelope glycoprotein, a cell comprising the vector or particle, and/or a composition comprising the particle of the same. In certain embodiments, the invention provides a cell comprising a chimeric antigen receptor (CAR) generated by the methods disclosed herein. In certain embodiments, the invention provides a producer cell comprising a vector comprising a Cocal vesiculovirus envelope glycoprotein and methods of making and using the cell.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition), Michael R. Green and Joseph Sambrook eds., and Harlow et al., Antibodies: A Laboratory Manual, Chapter 14, Cold Spring Harbor Laboratory, Cold Spring Harbor (2013, 2nd edition).

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

The term “antibody,” as used herein, refers to an immunoglobulin molecule which specifically binds with an antigen. Antibodies can be intact immunoglobulins derived from natural sources or from recombinant sources (i.e. a “Y” shaped structure or linked combinations thereof like the IgA dimer or IgM pentamer, each “Y” comprising two antigen binding sites, one at the end of each Fab region, each Fab region being each arm of the “Y”, each “Y” further comprising a Fc region, the Fc region being the base of the “Y”) and can be immunoreactive portions of intact immunoglobulins (i.e. an Fab region or a fragment thereof). Antibodies are often tetramers of immunoglobulin molecules. The antibodies in embodiments herein can exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

Furthermore, antigens can be derived from recombinant, mitochondrial, or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that embodiments herein include, but are not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA, and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when contacted with a cell causes a detectable level of change in a nucleic acid carried by the Cocal vesiculovirus envelope pseudotyped retroviral vector or a protein encoded by the nucleic acid, such as a CAR or a TCR.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B or T cell responses. An antigen can have one or more epitopes. Most antigens have many epitopes; i.e., they are multivalent. In general, an epitope is roughly about 10 amino acids or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue, or system.

The term “expand” as used herein refers to increasing in number, as in an increase in the number of cells (i.e. T cells). In one embodiment, the cells that are expanded ex vivo increase in number relative to the number originally present in the culture (i.e. a ten, one hundred, one thousand, ten thousand, hundred thousand, million, etc. increase in the number of T cells). In another embodiment, the cells that are expanded ex vivo increase in number relative to other cell types in the culture (i.e. a ten-fold increase in T cells relative to a 10% increase in other cell types). The term “ex vivo” as used herein refers to cells that have been removed from a living organism, (e.g., a human) and propagated outside the organism (e.g., in a culture dish, test tube, or bioreactor).

The term “expression” as used herein is defined as the transcription or translation of a particular nucleotide sequence driven by its promoter.

“Expression vector” refers to a vector comprising a recombinant polynucleotide comprising expression control sequences (i.e. transcription control sequences or promoters) operatively linked to a nucleotide sequence to be expressed (i.e. coding sequence). An expression vector comprises sufficient cis-acting elements for expression; other elements for expression can be supplied by the host cell or in an in vitro expression system. Expression vectors include all those known in the art, such as cosmids and plasmids (e.g., naked or contained in liposomes). Viruses (e.g., Sendai viruses, lentiviruses, retroviruses, adenoviruses, and adeno-associated viruses) that incorporate the recombinant polynucleotide can be considered a vector in that they carry the recombinant polynucleotide. Because the retroviral particles must be produced, the cells producing the retroviral particles can comprise expression vectors for the retrovirus particles that can be delivered as nucleic acid vectors (i.e. nucleofection of nucleic acids encoding the viral particle and the transgenes contained in the viral particle) or viral vectors (i.e. delivering by Sendai virus, the nucleic acids encoding the viral particle and the transgenes contained in the viral particle).

As used herein, a “host cell” is a cell transfected with a nucleic acid vector to replicate and produce more of the nucleic acid vector per se (i.e. more plasmid). Examples of host cells for plasmid and vector (nucleic acid vector) production includes bacteria, such as Escherichia coli.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical. A “modification” therefore, can refer to changes to the amino acid or nucleotide sequence so that the identity is no longer the same (i.e. amino acid substitutions, deletions, or additions). Generally, an addition or a deletion accounts for the shift in the amino acid or nucleotides (i.e. accounts for the “same position”) caused by the deletion or the addition by realigning the sequences after the addition or deletion so that those amino acids or nucleotides that are identical are aligned. For example, a deletion of 1 amino acid from a sequence of 10 will result in an amino acid sequence with 90% identity to the original sequence regardless of where the amino acid deletion is. A deletion of the first amino acid in 10 amino acid sequence will not result in a 0% identity because the new first amino acid will be aligned with the second amino acid in the reference sequence. In this regard, a deletion will be treated as a zero (i.e. a null or placeholder) at the position being deleted within the sequence for comparison, whereas an addition will be treated as a zero or a null at the position within the reference sequence.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to the genus of the same name in the Spumaretrovirinae subfamily of the Retroviridae family. Lentiviruses are unique among the retroviruses in being able to infect non-dividing cells; they can deliver a significant amount of genetic information into the DNA of the host cell, so they are one of the most efficient methods of a gene delivery viral vector. HIV, SIV, and FIV are all examples of lentiviruses. Viral vectors derived from lentiviruses offer the means to achieve significant levels of gene transfer in vivo.

The term “modified” as used herein, is meant a changed state or structure of a molecule or cell of the invention. Molecules may be modified in many ways, including chemically, structurally, and functionally. Cells may be modified through the introduction of nucleic acids.

The term “modulating,” as used herein, is meant mediating a detectable increase or decrease in the level of a response in a subject compared with the level of a response in the subject in the absence of a treatment or compound, or compared with the level of a response in an otherwise identical but untreated subject. The term encompasses perturbing or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in that “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, a “producer cell” is a cell transfected with nucleic acid vectors (i.e. plasmids or nucleic acid vectors) that are necessary and sufficient to produce a retroviral particle (including vectors that encode the Cocal vesiculovirus envelope protein), including optionally retroviral vectors carrying nucleic acids encoding genetic information that a cell is to be transduced or transfected with (i.e. a nucleic acid or vector encoding a chimeric antigen receptor (CAR)).

By the term “specifically binds,” as used herein with respect to an antibody or a CAR, is meant an antibody or CAR which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline, and murine mammals. Preferably, the subject is human.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “titer” refers to the concentration of a solution as determined by titration and in virology refers to the concentration of infectious viral particles in a solution, the concentration being obtained from a population of producer cells that have been infected with the virus or have been transfected with nucleic acids, nucleic acid vectors that encode the virus particle, or proteins that are necessary for the production of the virus particle. The titer obtained from the population of producer cells can be, optionally, concentrated therefrom, usually by centrifugation.

The term “therapeutic” as used herein means a treatment or prophylaxis. A therapeutic effect is obtained by suppression, remission, or eradication of a disease state.

The term “transfected,” “transformed,” or “transduced” as used herein refers to a process by which exogenous nucleic acid is transferred or introduced into the host cell. A “transfected,” “transformed,” or “transduced” cell is one that has been transfected, transformed, or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Vectors can be distinguished from one another (i.e. a plasmid versus a virus) by modifiers preceding “vector”, i.e. “a nucleic acid vector” versus “a viral vector,” i.e. whereby a “nucleic acid vector” encompasses a plasmid but not a virus, i.e. whereby a “viral vector” encompasses a virus but not a plasmid (the virus being understood to be an expression vector but not a plasmid per se or a nucleic acid vector per se even though it comprises a nucleic acid or a transgene). Examples of viral vectors include, but are not limited to, Sendai viral vectors, adenoviral vectors, adeno-associated virus vectors, retroviral vectors, which includes lentiviral vectors, and the like.

A “virus particle,” “viral particle,” or as sometimes used herein “particle” means a complete viral particle constituting the infective form of a virus and consisting of RNA or DNA (RNA in the case of a retrovirus) surrounded by a protein shell or envelope proteins. The protein shell or completed arrangement of the envelope proteins is known as a capsid, and it protects the interior core of the particle that includes the genetic information carried by the virus particle and other proteins, including in the case of non-inactivated proteins, those that are necessary for their replication, insertion, infection, or virulence. A “viral vector” or “vector particle” as used herein is a viral particle that comprises an isolated nucleic acid or transgene to be delivered to a target cell, the isolated nucleic acid either changing the genetics, epigenetics, or protein expression of the target cell, optionally, by encoding a chimeric antigen receptor to be expressed in the target cell. In the viral vector, the envelope protein can encapsulate the isolated nucleic acid or transgene to be delivered to the target cell.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Methods

The present invention provides compositions and methods for generating cells (e.g. T cells) comprising chimeric antigen receptors (CARs). In one aspect, the invention includes a method for delivering a nucleic acid sequence encoding a chimeric antigen receptor (CAR) to an immune cell or precursor cell thereof. The method comprises introducing into the immune cell or precursor cell thereof a transfer plasmid comprising a nucleotide sequence encoding a CAR, a retroviral vector comprising a nucleotide sequence encoding a Cocal vesiculovirus envelope protein, a plasmid comprising a nucleotide sequence encoding a retroviral Rev protein, and at least one plasmid comprising a nucleotide sequence encoding a retroviral Gag protein and a retroviral Pol protein. In certain embodiments, the amount of transfer plasmid introduced into the cell is higher than the amount of the retroviral vector comprising a nucleotide sequence encoding a Cocal vesiculovirus envelope protein.

The invention should be construed to include any chimeric antigen receptor (CAR) known in the art and those discussed in detail elsewhere herein.

In one aspect, the invention includes a method for generating a population of CAR T cells. The method comprises introducing into a T cell or precursor cell thereof, a transfer plasmid comprising a nucleotide sequence encoding a CAR, a retroviral vector comprising a nucleotide sequence encoding a Cocal vesiculovirus envelope protein, a plasmid comprising a nucleotide sequence encoding a retroviral Rev protein, and at least one plasmid comprising a nucleotide sequence encoding a retroviral Gag protein and a retroviral Pol protein. In certain embodiments, the amount of transfer plasmid introduced into the cell is higher than the amount of the retroviral vector comprising a nucleotide sequence encoding a Cocal vesiculovirus envelope protein.

In certain embodiments, the amount of transfer plasmid introduced is at least 2 times (×), 3×, 4×, 5×, 6×, 7×, 8×, 9×, 10×, or 20× the amount of the vector comprising a nucleotide sequence encoding a Cocal vesiculovirus envelope protein.

In certain embodiments, the Cocal vesiculovirus envelope protein is encoded by a nucleotide sequence at least 80%, at least 85%, at least 90%, at least 95%, at least 96%, at least 97%, at least 98%, at least 99%, or 100% identical to SEQ ID NO: 1. In certain embodiments, the Cocal vesiculovirus envelope protein comprises an amino acid sequence at least 80%, at least 85%, at least 90%, at least 95%, at least 96%, at least 97%, at least 98%, at least 99%, or 100% identical to SEQ ID NO: 2.

In certain embodiments, the expression of the envelope protein is under control of a transcriptional regulatory element. In certain embodiments, the transcriptional regulatory element is a eukaryotic promoter. In certain embodiments, the transcriptional regulatory element is a constitutive promoter.

In certain embodiments, the vector comprises a nucleotide sequence at least 80%, at least 85%, at least 90%, at least 95%, at least 96%, at least 97%, at least 98%, at least 99%, or 100% identical SEQ ID NO: 4.

In one aspect, a method for delivering a nucleic acid sequence into a heterogeneous population of immune cells is provided herein. The method comprises contacting the heterogeneous population of immune cells with a Cocal vesiculovirus envelope pseudotyped retroviral vector particle comprising the nucleic acid sequence. In some embodiments, the nucleic acid sequence encodes a CAR or a TCR. In some embodiments, the Cocal vesiculovirus envelope pseudotyped retroviral vector particle further comprises a Cocal vesiculovirus envelope protein encoded by the nucleic acid sequence set forth in SEQ ID NO:1. In some embodiments, the Cocal vesiculovirus envelope protein comprises the amino acid sequence of SEQ ID NO: 2.

In some embodiments, the immune cell or heterogeneous population of immune cells comprises a T cell. In some embodiments, the T cell comprises a CD8+ T cell. In some embodiments, the T cell comprises a CD4+ T cell. In some embodiments, the T cell comprises a regulatory T cell. In some embodiments, the heterogeneous population of immune cells comprises a CD8+ T cell or a CD4+ T cell. In some embodiments, the heterogeneous population of immune cells comprises a CD8+ T cell and a CD4+ T cell.

The methods disclosed herein can be scaled-up for batch production of cells comprising CARs. The cells can also be adapted for growth in suspension and/or to grow in serum-free cultures. The methods and compositions made by the methods (e.g. CAR T cells) can also be GMP compliant.

### C. Cocal vesiculovirus Envelope Glycoprotein and Particles Containing the Glycoprotein

The present invention provides compositions and methods for producing and using a Cocal vesiculovirus envelope glycoprotein, including: particles, such as viral particles, and cells, comprising the Cocal vesiculovirus envelope glycoprotein. The particles and Cocal vesiculovirus envelope glycoprotein have lower toxicity to cells producing them (i.e. “producer cells”) and higher transduction efficiencies of cells being infected by them (i.e. “target cells”). The invention also provides viral vector particles, which are particles that further comprise a nucleic acid transgene (e.g. a CAR) that is delivered to a cell during the infection of the cell by the virus. Nucleic acids and vectors encoding the Cocal vesiculovirus envelope glycoprotein are also included in the invention. Also included are producer cells comprising a nucleic acid or vector encoding the Cocal vesiculovirus envelope glycoprotein, the producer cells optionally producing the viral particles or viral vector particles.

In some embodiments, the viral particles can be self-inactivating. A self-inactivating viral particle can prevent viral transcription beyond the first round of viral replication. Consequently, a self-inactivating particle can be capable of infecting and the genetic information therein can be capable of integrating into a host genome (e.g., a mammalian genome) only once, and cannot be passed further. Accordingly, self-inactivating particles can greatly reduce the risk of creating a replication-competent virus.

In another aspect, a composition comprising the particles is provided. Since the particles and Cocal vesiculovirus envelope glycoprotein have lower toxicity to the cells producing them, the composition comprising the particles can have a higher titer of particles than compositions comprising other viral particles, including compositions comprising other retroviral particles and compositions comprising other retroviral particles having other envelope proteins, including VSV-G envelope proteins or first-generation, second-generation, or third-generation Cocal vesiculovirus envelope proteins. Without wishing to be bound by a particular theory, the higher viral titers appear to be due to the lower toxicity of the Cocal vesiculovirus envelope glycoprotein, nucleic acids or vectors encoding the same, and viral particles comprising the same, which allows the producer cells to make a higher concentration of viral particles than the same cells producing a particle comprising a VSV-G or first-generation, second-generation, or third-generation Cocal vesiculovirus envelope protein. Accordingly, the compositions have higher titers of mature and immature particles, higher titers of infective particles, and higher titers of genetic information carried within the particles (e.g. CARs) than compositions having particles that are enveloped by VSV-G or first-generation, second-generation, or third-generation Cocal vesiculovirus envelope proteins.

Since the particles and Cocal vesiculovirus envelope glycoprotein have lower toxicity to the cells producing them, the compositions comprising the particles can have a higher transduction efficiency than compositions comprising other viral particles, including compositions comprising other retroviral particles and compositions comprising other retroviral particles having other envelope proteins, including VSV-G envelope proteins or first-generation, second-generation, or third-generation Cocal vesiculovirus envelope proteins. Accordingly, the compositions have higher transduction efficiencies than compositions having particles that are enveloped by VSV-G or first-generation, second-generation, or third-generation Cocal vesiculovirus envelope proteins.

In another aspect, the Cocal vesiculovirus envelope glycoprotein is more effective at causing the particle or viral particle to enter a target cell. Accordingly, the compositions comprising the particles can have a higher transduction efficiency than compositions comprising other viral particles, including compositions comprising other retroviral particles and compositions comprising other retroviral particles having other envelope proteins, including VSV-G envelope proteins or first-generation, second-generation, or third-generation Cocal vesiculovirus envelope proteins. Accordingly, the compositions have higher transduction efficiencies than compositions having particles that are enveloped by VSV-G or first-generation, second-generation, or third-generation Cocal vesiculovirus envelope proteins.

In another aspect, since the particles and Cocal vesiculovirus envelope glycoprotein have lower toxicity, the particles and compositions comprising the particles can have a lower toxicity to cells and organisms (i.e. lower in vivo toxicity) being contacted with the particles or compositions compared with compositions comprising other viral particles, including compositions and particles comprising other retroviral particles, including other retroviral particles comprising VSV-G envelope proteins or first-generation, second-generation, or third-generation Cocal vesiculovirus envelope proteins. This lower toxicity to the cells and organisms can be measured by quantifying measures of toxicity for equivalent titers of particles. Alternatively, this lower toxicity to the cells and organism can be measured by having equivalent measures of toxicity but with the equivalent measure of toxicity being obtained from higher titers of the particles comprising the Cocal vesiculovirus envelope protein than particles comprising another envelope protein, such as VSV-G envelope proteins or first-generation, second-generation, or third-generation Cocal vesiculovirus envelope proteins.

Accordingly, the compositions of the present invention have higher transduction efficiencies compared with compositions having particles that are enveloped by VSV-G or first-generation, second-generation, or third-generation Cocal vesiculovirus envelope proteins. Without wishing to be bound to a particular theory, the higher transduction efficiencies could be due to the lower toxicity, in that if the toxicity is lower, then more cells that are infected with the viral particle will survive than cells infected with another viral particle, such as one enveloped by VSV-G or first-generation, second-generation, or third-generation Cocal vesiculovirus envelope proteins.

In certain embodiments, the Cocal vesiculovirus envelope protein comprises or consists of the amino acid sequence of SEQ ID NO: 2. In certain embodiments, the Cocal vesiculovirus envelope protein is at least 80%, at least 85%, at least 90%, at least 95%, at least 96%, at least 97%, at least 98%, at least 99%, or 100% identical to SEQ ID NO: 2. In certain embodiments, the Cocal vesiculovirus envelope protein amino acid sequence has from 1 to 10, 1 to 20, 1 to 30, 1 to 40, or 1 to 50 modifications (including additions, deletions, or substitutions) thereof.

In one aspect, an isolated Cocal vesiculovirus envelope protein is provided, the amino acid sequence of the protein being the amino acid sequence of SEQ ID NO: 2; an amino acid sequence with 90%-100%, 95%-100%, 96%-100%, 97%-100%, 98%-100%, 99%-100%, 90%-99%, 95%-99%, 96%-99%, 97%-99%, 98%-99%, or 99%-99.9% homology thereof; an amino acid sequence having from 1 to 10 amino acid modifications (including additions, deletions, or substitutions) thereof; an amino acid sequence having from 1 to 20 amino acid modifications thereof; an amino acid sequence having from 1 to 30 amino acid modifications thereof; an amino acid sequence having from 1 to 40 amino acid modifications thereof; an amino acid sequence having from 1 to 50 amino acid modifications thereof; an amino acid sequence having at least 1, 2, 3, 4, 5, 6, 7, 8, 9, 10, 20, 25, 30, 35, 40, 45, or 50 amino acid modifications thereof; or an amino acid sequence having less than 50, 45, 40, 35, 30, 25, 20, 15, 10, 9, 8, 7, 6, 5, 4, 3, 2, or 1 amino acid modifications thereof.

In one aspect, an isolated Cocal vesiculovirus envelope protein encoded by the nucleotide sequence of SEQ ID NO: 1, is provided. In certain embodiments, Cocal vesiculovirus envelope protein is encoded by a nucleotide sequence at least 80%, at least 85%, at least 90%, at least 95%, at least 96%, at least 97%, at least 98%, at least 99%, or 100% identical to SEQ ID NO: 1; a nucleotide sequence with 90%-100%, 95%-100%, 96%-100%, 97%-100%, 98%-100%, 99%-100%, 90%-99%, 95%-99%, 96%-99%, 97%-99%, 98%-99%, or 99%-99.9% homology thereof; a nucleotide sequence having from 1 to 10 base pair modifications (including additions, deletions, or substitutions) thereof; a nucleotide sequence having from 1 to 20 base pair modifications thereof; a nucleotide sequence having from 1 to 30 base pair modifications thereof; a nucleotide sequence having from 1 to 40 base pair modifications thereof; a nucleotide sequence having from 1 to 50 base pair modifications thereof; a nucleotide sequence having at least 1, 2, 3, 4, 5, 6, 7, 8, 9, 10, 20, 25, 30, 35, 40, 45, or 50 base pair modifications thereof; or a nucleotide sequence having less than 50, 45, 40, 35, 30, 25, 20, 15, 10, 9, 8, 7, 6, 5, 4, 3, 2, or 1 base pair modifications thereof.

The lower toxicity of the Cocal vesiculovirus envelope glycoprotein or the particles containing said protein on the producer cells can be indicated by: 1) having greater total producer cell numbers, 2) greater numbers of producer cells that express a positive marker or reporter gene for transfection of the plasmids encoding the particles (i.e. the nucleic acid or nucleic acid vector encoding the Cocal vesiculovirus envelope glycoprotein further encodes GFP so that the transfected cells fluoresce, or the nucleic acid encapsulated by the viral vector particle encodes a reporter gene such as GFP, so that the infected cells fluoresce after infection), 3) greater levels of the positive marker or reporter gene within each cell transfected (i.e. if GFP is a positive marker or reporter gene then greater mean fluorescence per cell), 4) lower measures of cell death, or 5) higher functional titers, numbers, or ratios of infectious particles to nonfunctional or immature particles within the producer cells compared to the same measures from producer cells that express a different envelope protein or a particle (i.e. VSV-G or first-generation, second-generation, or third-generation Cocal vesiculovirus envelope glycoprotein).

The measures of cell death in the cells, including in producer cells and cells infected with the virus particles, can be 10, 20, 30, 40, 50, 60, 70, 80, 90, 100, 10³, 10⁴, 10⁵, 10⁶, 10⁷, or 10⁸ fold lower than the same cells which are, instead, transfected to express a VSV-G enveloped particle or are infected with the same virus that instead comprises VSV-G. The measures of cell death in the cells, including in producer cells and cells infected with the virus particles, in some embodiments are 10, 20, 30, 40, 50, 60, 70, 80, 90, 100, 10³, 10⁴, 10⁵, 10⁶, 10⁷, or 10⁸ fold lower than the same cells which are, instead, transfected to express another Cocal vesiculovirus envelope glycoprotein containing particle (e.g. first-generation, second-generation, or third-generation particles or glycoproteins) or are infected with the same virus that instead comprises another Cocal vesiculovirus envelope glycoprotein.

The total number of cells, including producer cells and cells infected with the virus particles, can be 10, 20, 30, 40, 50, 60, 70, 80, 90, 100, 10³, 10⁴, 10⁵, 10⁶, 10⁷, or 10⁸ fold greater than the same cells that are, instead, transfected to express a VSV-G enveloped particle or are infected with the same virus that instead comprises a VSV-G. In some embodiments, the total number of the cells, including in producer cells and cells infected with the virus particles, are 10, 20, 30, 40, 50, 60, 70, 80, 90, 100, 10³, 10⁴, 10⁵, 10⁶, 10⁷, or 10⁸ fold greater than the same cells which are, instead, transfected to express another Cocal vesiculovirus envelope glycoprotein containing particle (e.g. first-generation, second-generation, or third-generation particles or glycoproteins) or are infected with the same virus that instead comprises another Cocal vesiculovirus envelope glycoprotein (e.g. first-generation, second-generation, or third-generation glycoproteins).

In some embodiments, the number of producer cells expressing the positive marker or reporter gene for the transfection of the components encoding the viral particles are 10, 20, 30, 40, 50, 60, 70, 80, 90, 100, 10³, 10⁴, 10⁵, 10⁶, 10⁷, or 10⁸ fold greater than the same producer cells which are, instead, transfected to express a VSV-G enveloped particle. In some embodiments, the number of producer cells expressing the positive marker or reporter gene for the transfection of the components encoding the viral particles are 10, 20, 30, 40, 50, 60, 70, 80, 90, 100, 10³, 10⁴, 10⁵, 10⁶, 10⁷, or 10⁸ fold greater than the same producer cells which are, instead, transfected to express another Cocal vesiculovirus envelope glycoprotein containing particle (e.g. first-generation, second-generation, or third-generation particles or glycoproteins). In some embodiments, the number of cells expressing the positive marker or reporter gene on the transgene within the viral particle are 10, 20, 30, 40, 50, 60, 70, 80, 90, 100, 10³, 10⁴, 10⁵, 10⁶, 10⁷, or 10⁸ fold greater than the same cells which are, instead, infected with a VSV-G enveloped particle. In some embodiments, the number of cells expressing the positive marker or reporter gene on the transgene within the viral particle are 10, 20, 30, 40, 50, 60, 70, 80, 90, 100, 10³, 10⁴, 10⁵, 10⁶, 10⁷, or 10⁸ fold greater than the same producer cells which are, instead, infected with another Cocal vesiculovirus envelope glycoprotein containing particle (e.g. first-generation, second-generation, or third-generation particles or glycoproteins).

In some embodiments, the amount of the positive marker for the transfection of the components encoding the viral particles are 10, 20, 30, 40, 50, 60, 70, 80, 90, 100, 10³, 10⁴, 10⁵, 10⁶, 10⁷, or 10⁸ fold greater per transfected cell than the same producer cells which are, instead, transfected to express a VSV-G enveloped particle. In some embodiments, the amount of the positive marker for the transfection of the components encoding the viral particles are 10, 20, 30, 40, 50, 60, 70, 80, 90, 100, 10³, 10⁴, 10⁵, 10⁶, 10⁷, or 10⁸ fold greater per transfected cell than the same producer cells which are, instead, transfected to express another Cocal vesiculovirus envelope glycoprotein containing particle (e.g. first-generation, second-generation, or third-generation particles or glycoproteins).

In some embodiments, the amount of the positive marker from the transgene within the infected cell are 10, 20, 30, 40, 50, 60, 70, 80, 90, 100, 10³, 10⁴, 10⁵, 10⁶, 10⁷, or 10⁸ fold greater per infected cell than the same cells which are, instead, infected by a particle encapsulated by a VSV-G enveloped particle. In some embodiments, the amount of the positive marker from the transgene within the infected cell are 10, 20, 30, 40, 50, 60, 70, 80, 90, 100, 10³, 10⁴, 10⁵, 10⁶, 10⁷, or 10⁸ fold greater per transfected cell than the same infected cell which are, instead, transfected to express another Cocal vesiculovirus envelope glycoprotein containing particle (e.g. first-generation, second-generation, or third-generation particles or glycoproteins).

In some embodiments, the number of cells expressing a chimeric antigen receptor (CAR) is 10, 20, 30, 40, 50, 60, 70, 80, 90, 100, 10³, 10⁴, 10⁵, 10⁶, 10⁷, or 10⁸ fold greater per infected cell than the same cells which are, instead, infected by a particle encapsulated by a VSV-G enveloped particle. In some embodiments, the number of cells expressing a chimeric antigen receptor (CAR) is 10, 20, 30, 40, 50, 60, 70, 80, 90, 100, 10³, 10⁴, 10⁵, 10⁶, 10⁷, or 10⁸ fold greater per infected cell than the same infected cells which are, instead, transfected to express another Cocal vesiculovirus envelope glycoprotein containing particle (e.g. first-generation, second-generation, or third-generation particles or glycoproteins).

In some embodiments, the particle titer is higher than the viral titers from the same producer cells that are, instead, transfected with nucleic acid vectors and nucleic acids encoding VSV-G enveloped particles or Cocal vesiculovirus envelope glycoprotein containing particle (e.g. first-generation, second-generation, or third-generation particles or glycoproteins) because the nucleic acid vectors and nucleic acids encoding the particles and the particles themselves and the envelope glycoproteins themselves are less toxic to the producer cells. In some embodiments, the particle titers are 10, 20, 30, 40, 50, 60, 70, 80, 90, 100, 10³, 10⁴, 10⁵, 10⁶, 10⁷, or 10⁸ fold greater than from the same producer cells which are, instead, transfected to express a VSV-G enveloped particle or another Cocal vesiculovirus envelope glycoprotein containing particle (e.g. first-generation, second-generation, or third-generation particles or glycoproteins).

In some embodiments, the percent of infectious particles to total particles (including non-infectious or immature particles) are higher in the cells producing the particles than the same percentage in the same cells that are, instead, transfected with nucleic acid vectors and nucleic acids encoding VSV-G enveloped particles or Cocal vesiculovirus envelope glycoprotein containing particle (e.g. first-generation, second-generation, or third-generation particles or glycoproteins) because the nucleic acid vectors and nucleic acids encoding the particles and the particles themselves and the envelope glycoproteins themselves are less toxic to the producer cells. In some embodiments the percent of infectious particles is 2×, 3×, 4×, 5×, 6×, 7×, 8×, 9×, 10×, 30×, 100×, 300×, 1000× higher than the percent of infectious particles from the same cells that are, instead, transfected with nucleic acid vectors and nucleic acids encoding VSV-G enveloped particles or Cocal vesiculovirus envelope glycoprotein containing particle (e.g. first-generation, second-generation, or third-generation particles or glycoproteins).

In some embodiments, the transduction efficiency of the particle comprising the codon optimized Cocal vesiculovirus envelope glycoprotein is higher than particles comprising a VSV-G envelope glycoprotein or first-generation, second-generation, or third-generation Cocal vesiculovirus envelope glycoprotein. In some embodiments, transduction efficiency of the particle is 5%, 10%, 15%, 20%, 25%, 30%, 35%, 40%, 45%, 50%, 55%, 60%, 65%, 70%, 75%, 80%, 85%, 90%, 95%, 2×, 3×, 4×, 5×, 6×, 7×, 8×, 9×, 10×, 30×, 100×, 300×, 1000× higher than particles comprising a VSV-G envelope glycoprotein or Cocal vesiculovirus envelope glycoprotein (e.g. first-generation, second-generation, or third-generation glycoproteins).

In some embodiments, the transduction efficiency of the composition comprising the particle comprising the codon optimized Cocal vesiculovirus envelope glycoprotein is higher than that of compositions comprising particles comprising a VSV-G envelope glycoprotein or first-generation, second-generation, or third-generation Cocal vesiculovirus envelope glycoprotein. In some embodiments, transduction efficiency of the compositions comprising the particle is 5%, 10%, 15%, 20%, 25%, 30%, 35%, 40%, 45%, 50%, 55%, 60%, 65%, 70%, 75%, 80%, 85%, 90%, 95%, 2×, 3×, 4×, 5×, 6×, 7×, 8×, 9×, 10×, 30×, 100×, 300×, 1000× higher than that of compositions comprising a particle comprising a VSV-G envelope glycoprotein or Cocal vesiculovirus envelope glycoprotein (e.g. first-generation, second-generation, or third-generation glycoproteins). In some embodiments, the transduction efficiency is measured by infecting the same cells with the same titer and measuring the number of cells infected. In some embodiments the transduction efficiency is measured by infecting the same cells with different titers and determining which titer achieves the same percentage of infected cells.

In such embodiments, the target cells from which the transduction efficiency is measured can be the same across compositions (i.e. the compositions of particles presently disclosed compared to the compositions of particles comprising or encapsulated by VSV-G envelope glycoprotein or first-generation, second-generation, or third-generation Cocal vesiculovirus envelope glycoproteins). In certain embodiments, the target cells from which the transduction efficiency is measured are HEK293-T cells. In some embodiments, the transduction efficiency is determined from the same amount of protein obtained from the producer cells used to produce the embodied compositions comprising the embodied particles and compared to the compositions containing known particles obtained from the same producer cells, transfected under the same conditions. In some embodiments, the transduction efficiency is determined from the same volume of supernatant obtained from the producer cells used to produce the embodied compositions comprising the embodied particles and compared to the compositions containing known particles obtained from the same producer cells, transfected under the same conditions. In some embodiments, this volume is centrifuged under conditions that cause the viral particles to pellet and then is resuspended in the same volume, thus concentrating the embodied particles and thus identically concentrating the known particles (i.e. particles containing or enveloped by VSV-G envelope glycoprotein or first-generation, second-generation, or third-generation Cocal vesiculovirus envelope glycoproteins) for comparison of the transduction efficiency. In some embodiments, said volume of the composition of embodied particles is concentrated (i.e. by lyophilization, evaporation, etc.) and the composition of the known particles (i.e. particles containing or enveloped by VSV-G envelope glycoprotein or first-generation, second-generation, or third-generation Cocal vesiculovirus envelope glycoproteins) are concentrated in an identical manner to provide for a comparison of the transduction efficiency.

Methods of centrifuging or concentrating viruses can be found in, for example, MOLECULAR CLONING: A LABORATORY MANUAL (Joseph F. Sambrook and David W. Russell, eds.; 3rd Ed.; Vols. 1, 2, and 3; Cold Spring Harbor Laboratory Press; 2001) and MOLECULAR CLONING: A LABORATORY MANUAL (Michael R. Green and Joseph F. Sambrook, eds.; 4th Ed.; Vols. 1, 2, and 3; Cold Spring Harbor Laboratory Press; 2012), which are incorporated by reference.

In certain embodiments, the retrovirus particle comprising the Cocal vesiculovirus envelope protein includes but is not limited to order Ortervirales, including Belpaoviridae, Metaviridae, Pseudoviridae, Retroviridae (e.g. HIV), Caulimoviridae (e.g. a VII group virus family); subfamily Orthoretrovirinae, which includes genera Alpharetrovirus, Betaretrovirus, Gammaretrovirus, Deltaretrovirus, Epsilonretrovirus, Lentivirus; subfamily Spumaretrovirinae, which includes genera Bovispumavirus, Equispumavirus, Felispumavirus, Prosimiispumavirus, Simiispumavirus. Preferred embodiments include Orthoretrovirinae, Alpharetrovirus, Betaretrovirus, Deltaretrovirus, Epsilonretrovirus, Gammaretrovirus, Lentivirus, Spumaretrovirinae, Bovispumavirus, Equispumavirus, Felispumavirus, Prosimiispumavirus, and Simiispumavirus particles. In some embodiments, the retrovirus particle is derived from elements, proteins, and enzymes from different members of the orders, families, subfamilies, genera described supra. In this regard, in some embodiments the particle is not limited to one single subfamily or genus, but can be comprised of elements, proteins, enzymes, and nucleic acids from multiple different subfamilies or genera. In other embodiments, the elements, proteins, enzymes, and nucleic acids are from the same family, same subfamily, or same genera.

In certain embodiments, the retroviral particles containing the Cocal vesiculovirus envelope protein comprise lentiviral vectors, being lentiviruses or being derived from lentiviruses, including the Human Immunodeficiency Viruses (HIV-1, HIV-2) and the Simian Immunodeficiency Virus (SIV). Lentiviral vectors are capable of infecting non-dividing cells and can be used for both in vivo and ex vivo gene transfer and expression, e.g., of a nucleic acid encoding a CAR (see, e.g., U.S. Pat. No. 5,994,136).

### D. Chimeric Antigen Receptors

The present invention provides compositions and methods for modified immune cells or precursors thereof, e.g., modified T cells, comprising a chimeric antigen receptor (CAR). In some embodiments, the immune cell has been genetically modified to express the CAR by being transduced with the Cocal vesiculovirus envelop pseudotyped retroviral vectors carrying the genetic information (i.e. transgene) encoding the CAR. CARS herein comprise an antigen-binding domain, a transmembrane domain, and an intracellular domain.

The antigen-binding domain can be operably linked to another domain of the CAR, such as the transmembrane domain or the intracellular domain, both described elsewhere herein, for expression in the cell. In one embodiment, a first nucleic acid sequence encoding the antigen-binding domain is operably linked to a second nucleic acid encoding a transmembrane domain, and further operably linked to a third a nucleic acid sequence encoding an intracellular domain.

The antigen-binding domains described herein can be combined with any of the transmembrane domains described herein, any of the intracellular domains or cytoplasmic domains described herein, or any of the other domains described herein that can be included in a CAR. A subject CAR herein can also include a hinge domain as described herein. A subject CAR herein can also include a spacer domain as described herein. In some embodiments, each of the antigen-binding domain, transmembrane domain, and intracellular domain is separated by a linker.

### Antigen-Binding Domain

The antigen-binding domain of a CAR is an extracellular region of the CAR for binding to a specific target antigen including proteins, carbohydrates, and glycolipids. In some embodiments, the CAR comprises affinity to a target antigen on a target cell. The target antigen can include any type of protein, or epitope thereof, associated with the target cell. For example, the CAR can comprise affinity to a target antigen on a target cell that indicates a particular disease state of the target cell.

In certain embodiments, the target cell antigen is a tumor associated antigen (TAA). Examples of tumor associated antigens (TAAs), include but are not limited to, differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS. In a preferred embodiment, the antigen binding domain of the CAR targets an antigen that includes but is not limited to CD19, CD20, CD22, ROR1, Mesothelin, CD33/IL3Ra, c-Met, PSMA, PSCA, Glycolipid F77, EGFRvIII, GD-2, NY-ESO-1 TCR, MAGE A3 TCR, EGFR, IL-13Ra2, Folate receptor-alpha, TnMUC1, glypican-2 (GPC2), cancer cell-associated GPC2, Mucin 1 (MUC-1), and Glycosyl-phosphatidylinositol (GPI)-linked GDNF family α-receptor 4 (GFRα4; GFRalpha4), and the like.

In certain embodiments, the target cell antigen is CD4.

Depending on the desired antigen to be targeted, the CAR can be engineered to include the appropriate antigen-binding domain that is specific to the desired antigen target. For example, if CD19 is the desired antigen that is to be targeted, an antibody for CD19 can be used as the antigen bind moiety for incorporation into the CAR.

As described herein, a CAR of the present disclosure having affinity for a specific target antigen on a target cell can comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin.

In some embodiments, a CAR of the present disclosure can have affinity for one or more target antigens on one or more target cells. In some embodiments, a CAR can have affinity for one or more target antigens on a target cell. In such embodiments, the CAR is a bispecific CAR, or a multi-specific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains can be arranged in tandem and can be separated by linker peptides. For example, in a CAR comprising two target-specific binding domains, the binding domains are connected to each other covalently on a single polypeptide chain, through an oligo- or polypeptide linker, an Fc hinge region, or a membrane hinge region.

The antigen-binding domain can include any domain that binds to the antigen and can include, but is not limited to, a monoclonal antibody, a polyclonal antibody, a synthetic antibody, a human antibody, a humanized antibody, a non-human antibody, and any fragment thereof. In some embodiments, the antigen-binding domain portion comprises a mammalian antibody or a fragment thereof. The choice of antigen-binding domain can depend upon the type and number of antigens that are present on the surface of a target cell.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen-binding domain (e.g., CD19 binding domain) comprises an scFv having the configuration from N-terminus to C-terminus, VH-linker-VL. In some embodiments, the antigen-binding domain comprises an scFv having the configuration from N-terminus to C-terminus, VL-linker-VH. Those of skill in the art would be able to select the appropriate configuration for use herein.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO: 5), (GGGS)n (SEQ ID NO: 6), and (GGGGS)n (SEQ ID NO: 7), where n represents an integer of at least 1. Exemplary linker sequences can comprise amino acid sequences including, without limitation, GGSG (SEQ ID NO: 8), GGSGG (SEQ ID NO: 9), GSGSG (SEQ ID NO: 10), GSGGG (SEQ ID NO: 11), GGGSG (SEQ ID NO: 12), GSSSG (SEQ ID NO: 13), GGGGS (SEQ ID NO: 14), GGGGSGGGGSGGGGS (SEQ ID NO: 15) and the like. Those of skill in the art would be able to select the appropriate linker sequence for use herein. In one embodiment, an antigen-binding domain comprises a heavy chain variable region (VH) and a light chain variable region (VL), wherein the VH and VL is separated by the linker sequence having the amino acid sequence GGGGSGGGGSGGGGS (SEQ ID NO:15), which can be encoded by the nucleic acid sequence GGTGGCGGTGGCTCGGGCGGTGGTGGGTCGGGTGGCGGCGGATCT (SEQ ID NO:16).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27(6):455-51; Peter et al., J Cachexia Sarcopenia Muscle 2012 Aug. 12; Shieh et al., J. Imunol. 2009 183(4):2277-85; Giomarelli et al., Thromb Haemost 2007 97(6):955-63; Fife et al., J. Clin. Invst. 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J. Biol. Chem. 2003 25278(38):36740-7; Xie et al., Nat. Biotech. 1997 15(8):768-71; Ledbetter et al., Crit. Rev. Immunol. 1997 17(5-6):427-55; Ho et al., BioChim. Biophys. Acta. 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen-binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In some embodiments, the antigen-binding domain can be derived from the same species in which the CAR will ultimately be used. For example, for use in humans, the antigen-binding domain of the CAR can comprise a human antibody or a fragment thereof. In some embodiments, the antigen-binding domain can be derived from a different species in which the CAR will ultimately be used. For example, for use in humans, the antigen-binding domain of the CAR can comprise a murine antibody or a fragment thereof

### Transmembrane Domain

CARs herein can comprise a transmembrane domain that connects the antigen-binding domain of the CAR to the intracellular domain of the CAR. The transmembrane domain of a subject CAR is a region that is capable of spanning the plasma membrane of a cell (e.g., an immune cell or precursor thereof). In some embodiments, the transmembrane domain is for insertion into a cell membrane, e.g., a eukaryotic cell membrane. In some embodiments, the transmembrane domain is interposed between the antigen-binding domain and the intracellular domain of a CAR.

In some embodiments, the transmembrane domain is naturally associated with one or more of the domains in the CAR. In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain can be derived either from a natural or a synthetic source. Where the source is natural, the domain can be derived from any membrane-bound or transmembrane protein, e.g., a Type I transmembrane protein. Where the source is synthetic, the transmembrane domain can be any artificial sequence that facilitates insertion of the CAR into a cell membrane, e.g., an artificial hydrophobic sequence. Examples of the transmembrane domain of particular use herein include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), 4-1BB (CD137), ICOS (CD278), CD154 (CD40L), Toll-like receptor 1 (TLR1), TLR2, TLR3, TLR4, TLR5, TLR6, TLR7, TLR8, TLR9, and a transmembrane domain derived from a killer immunoglobulin-like receptor (KIR) (also known as killer-cell immunoglobulin-like receptors). In some embodiments, the transmembrane domain can be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen-binding domains described herein, any of the intracellular domains described herein, or any of the other domains described herein that can be included in a subject CAR.

In some embodiments, the transmembrane domain further comprises a hinge region. A subject CAR herein can also include a hinge region. The hinge region of the CAR is a hydrophilic region which is located between the antigen-binding domain and the transmembrane domain. In some embodiments, this domain facilitates proper protein folding for the CAR. The hinge region is an optional component for the CAR. The hinge region can include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which can be as small as, three glycines (Gly), as well as CH1 and CH3 domains of IgGs (such as human IgG4).

In some embodiments, a subject CAR of the present disclosure includes a hinge region that connects the antigen-binding domain with the transmembrane domain, which, in turn, connects to the intracellular domain. The hinge region is preferably capable of supporting the antigen-binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen-binding domain to have a structure to optimally recognize the specific structure and density of the target antigens on a cell such as tumor cell (Hudecek et al., supra). The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than 20 amino acids (e.g., 30, 40, 50, 60 or more amino acids).

For example, hinge regions include glycine polymers (G)ₙ, glycine-serine polymers (including, for example, (GS)ₙ, (GSGGS)ₙ (SEQ ID NO:5) and (GGGS)ₙ (SEQ ID NO:6), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequences including, but not limited to, GGSG (SEQ ID NO:8), GGSGG (SEQ ID NO:9), GSGSG (SEQ ID NO:10), GSGGG (SEQ ID NO:11), GGGSG (SEQ ID NO:12), GSSSG (SEQ ID NO:13), and the like.

In some embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87(1):162-166; and Huck et al., Nucleic Acids Res. (1986) 14(4): 1779-1789. As non-limiting examples, an immunoglobulin hinge region can include one of the following amino acid sequences: DKTHT (SEQ ID NO:17); CPPC (SEQ ID NO:18); CPEPKSCDTPPPCPR (SEQ ID NO:19) (see, e.g., Glaser et al., J. Biol. Chem. (2005) 280:41494-41503); ELKTPLGDTTHT (SEQ ID NO:20); KSCDKTHTCP (SEQ ID NO:21); KCCVDCP (SEQ ID NO:22); KYGPPCP (SEQ ID NO:23); EPKSCDKTHTCPPCP (SEQ ID NO:24) (human IgG1 hinge); ERKCCVECPPCP (SEQ ID NO:25) (human IgG2 hinge); ELKTPLGDTTHTCPRCP (SEQ ID NO:26) (human IgG3 hinge); SPNMVPHAHHAQ (SEQ ID NO:27) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO:28); see, e.g., Yan et al., J. Biol. Chem. (2012) 287: 5891-5897. In one embodiment, the hinge region can comprise an amino acid sequence derived from human CD8, or a variant thereof.

### Intracellular Signaling Domain

A subject CAR herein also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

Examples of an intracellular domain for use herein include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5, and CD28. In one embodiment, the intracellular signaling domain can be human CD3 zeta chain, FcγRIII, FcsRI, cytoplasmic tails of Fc receptors, an immunoreceptor tyrosine-based activation motif (ITAM) bearing cytoplasmic receptors, and combinations thereof.

In one embodiment, the intracellular signaling domain of the CAR includes any portion or the whole of one or more co-stimulatory molecules, such as at least one signaling domain from a TNFR superfamily protein, CD27, CD28, 4-1BB (CD137), OX40 (CD134), PD-1, CD7, LIGHT, CD83L, DAP10, DAP12, CD27, CD2, CDS, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR), CD2, CD3, CD8, or a derivative or variant thereof, any synthetic sequence thereof that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CDlib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains can include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, and FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceRI gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in a CAR of the present disclosure includes a DAP10/CD28 type signaling chain. In one embodiment, an intracellular signaling domain suitable for use in a CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion can be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the antigen-binding domains described herein, any of the transmembrane domains described herein, or any of the other domains described herein that can be included in the CAR.

### E. Sources of Immune Cells

In some embodiments, a source of immune cells or a heterologous population thereof is obtained from a subject for ex vivo manipulation. Sources of target cells for ex vivo manipulation can also include, e.g., autologous or heterologous donor blood, cord blood, or bone marrow. For example the source of immune cells can be from the subject to be treated with the modified immune cells after having been contacted with the retroviral particle, e.g., the subject's blood, the subject's cord blood, or the subject's bone marrow. Non-limiting examples of subjects include humans, dogs, cats, mice, rats, and transgenic species thereof. Preferably, the subject is a human.

Immune cells and heterologous populations thereof can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and NK cells. Other exemplary cells include stem cells, such as multipotent and pluripotent stem cells, including induced pluripotent stem cells (iPSCs). In some aspects, the cells are human cells. With reference to the subject to be treated, the cells can be allogeneic or autologous. The cells typically are primary cells, such as those isolated directly from a subject or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the heterologous population of cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, can be used.

In some embodiments, the methods include isolating immune cells from the subject, preparing, processing, culturing, or engineering them. In some embodiments, preparation of the engineered cells includes one or more culture or preparation steps. The cells for engineering as described can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample containing a heterologous population of cells is blood or a blood-derived sample, or is or is derived from an apheresis or leukapheresis product. Exemplary heterologous population of cells include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. The heterologous population of cells include, in the context of cell therapy, e.g., adoptive cell therapy, samples from autologous and allogeneic sources.

In some embodiments, the heterologous population of cells are derived from cell lines, e.g., T cell lines. The heterologous population of cells in some embodiments are obtained from a xenogeneic source, for example, from mouse, rat, non-human primate, and pig. In some embodiments, isolation of the heterologous population of cells includes one or more preparation or non-affinity based cell separation steps. In some examples, the heterologous population of cells are washed, centrifuged, and/or incubated in the presence of one or more reagents, for example, to remove unwanted components, enrich for desired components, lyse or remove cells sensitive to particular reagents. In some examples, the heterologous population of cells are separated based on one or more property, such as density, adherent properties, size, sensitivity, or resistance to particular components.

In some examples, the heterologous population of cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the heterologous population of cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, the heterologous population of immune cells are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The heterologous population of cells collected by apheresis can be washed to remove the plasma fraction and to place the cells in an appropriate buffer or media, such as phosphate buffered saline (PBS) or wash solution lacks calcium and can lack magnesium or can lack many if not all divalent cations, for subsequent processing steps. After washing, the heterologous population of immune cells can be resuspended in a variety of biocompatible buffers, such as, for example, Ca-free, Mg-free PBS. Alternatively, the undesirable components of the apheresis sample can be removed and the cells directly resuspended in culture media.

In some embodiments, the methods include the separation of different cell types based on the expression or presence in the cell of one or more specific molecules, such as surface markers, e.g., surface proteins, intracellular markers, or nucleic acid. In some embodiments, any known method for separation based on such markers can be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD 14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated or cultured prior to, during, or after being contacted with the Cocal vesiculovirus enveloped pseudotyped retroviral particle. The incubation steps can include culture, cultivation, stimulation, activation, or propagation. In some embodiments, the compositions or cells are incubated in the presence of stimulating conditions or a stimulatory agent. Such conditions include those designed to induce proliferation, expansion, activation, or survival of cells in the population, to mimic antigen exposure, or to prime the cells for contact with the Cocal vesiculovirus enveloped pseudotyped retroviral particle and introduction of the transgene contained therein, including introduction with the nucleic acids and nucleic acid vectors encoding the CAR or TCR. The conditions can include one or more of particular media, temperature, oxygen content, carbon dioxide content, time, agents, e.g., nutrients, amino acids, antibiotics, ions, or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, or one or more cytokines. Optionally, the expansion method can further comprise the step of adding anti-CD3 or anti CD28 antibody to the culture medium (e.g., at a concentration of at least about 0.5 ng/ml) or a genetically modified T cell to express a CAR comprising an anti-CD3 antigen binding domain. In some embodiments, the stimulating agents include IL-2 or IL-15, for example, an IL-2 concentration of at least about 10 units/mL.

In another embodiment, heterologous population of immune cells and optionally, the T cells therein are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells therein can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies against one or more of CD14, CD20, CD11b, CD16, HLA-DR, and CD8.

For isolation of a desired population of cells by positive or negative selection, the concentration of cells and surface (e.g., particles such as beads) can be varied. In certain embodiments, it can be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells can be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing can be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to being contacted with the Cocal vesiculovirus envelope pseudotyped retroviral vector particle. Methods for isolating Tregs are described in U.S. Pat. Nos. 7,754,482, 8,722,400, and 9,555,105, and U.S. patent application Ser. No. 13/639,927, contents of which are incorporated herein in their entirety.

Whether prior to or after Cocal vesiculovirus envelope pseudotyped retroviral vector particle comprising a nucleic acid vector encoding a TCR or CAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the heterologous population of immune cells or T cells can be expanded by contact with a surface having attached thereto an agent that stimulates a CD3/TCR complex associated signal and a ligand that stimulates a co-stimulatory molecule on the surface of the T cells. In particular, T cell populations can be stimulated by contact with an anti-CD3 antibody, or antigen-binding fragment thereof, a T cell expressing a CAR comprising an anti-CD3 antigen binding domain, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) and these can be used in the method, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium can be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the method includes cryopreserving the expanded T cells. The cryopreserved T cells are thawed prior to introducing nucleic acids into the T cell.

In another embodiment, the method further comprises isolating T cells and expanding the T cells. In another embodiment, the method further comprises cryopreserving the T cells prior to expansion. In yet another embodiment, the cryopreserved T cells are thawed prior to contacting the T cells with the Cocal vesiculovirus envelope pseudotyped retroviral vector particle.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times can be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there can be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells can be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that can contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells can include an agent that can co-stimulate the T cells. For example, an agent that can stimulate CD3 is an antibody to CD3, and an agent that can stimulate CD28 is an antibody to CD28. A cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more. In one embodiment, human T regulatory cells are expanded via anti-CD3 antibody coated KT64.86 artificial antigen presenting cells (aAPCs). Methods for expanding and activating T cells can be found in U.S. Pat. Nos. 7,754,482, 8,722,400, and 9,555,105, contents of which are incorporated herein in their entirety.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation can include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent can stimulate the T cells, such as by stimulating further expansion, effector function, or another T cell function.

In some embodiments, the cells being transduced by the Cocal vesiculovirus envelope pseudotyped retroviral vector particle make up at least 50%, 60%, 70%, 80%, 90%, 91%, 92%, 93%, 94%, 95%, 96%, 97%, 98%, 99%, or more of the total cells in the heterologous population of immune cells. In some embodiments, the cells being transduced by the Cocal vesiculovirus envelope pseudotyped retroviral vector particle make up at least 50%, 60%, 70%, 80%, 90%, 91%, 92%, 93%, 94%, 95%, 96%, 97%, 98%, 99%, or more the T cells or CD8+ or CD4+ cells or regulatory T cells within the heterologous population of T cells.

In some embodiments, the cells comprising a CAR make up at least 50%, 60%, 70%, 80%, 90%, 91%, 92%, 93%, 94%, 95%, 96%, 97%, 98%, 99%, or more of the total cells in the heterologous population of immune cells. In some embodiments, the cells comprising a CAR make up at least 50%, 60%, 70%, 80%, 90%, 91%, 92%, 93%, 94%, 95%, 96%, 97%, 98%, 99%, or more the T cells or CD8+ or CD4+ cells or regulatory T cells within the heterologous population of T cells.

In some embodiments, the ratio of Cocal vesiculovirus envelope pseudotyped retroviral vector particle to cells being transduced by the particles is at least 1000:1, 333:1, 100:1, 33:1, 10:1, 3:1, 1:1, 1:3, 1:10, 1:33, 1:100, 1:333, 1:1000. In some embodiments, the ratio of Cocal vesiculovirus envelope pseudotyped retroviral vector particle to cells being transduced by the particles is no more than 1000:1, 333:1, 100:1, 33:1, 10:1, 3:1, 1:1, 1:3, 1:10, 1:33, 1:100, 1:333, 1:1000.

In certain embodiments, heterologous populations of immune cells or optionally the T cells therein, are contacted with the Cocal vesiculovirus envelope pseudotyped retroviral vector particle in population sizes that range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges. In certain embodiments, heterologous populations of immune cells or optionally the T cells therein, are contacted with a total of 1×10⁹ Cocal vesiculovirus envelope pseudotyped retroviral vector particle in cell population sizes that range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges. In certain embodiments, heterologous populations of immune cells or optionally the T cells therein, are contacted with a total of 1×10⁸ Cocal vesiculovirus envelope pseudotyped retroviral vector particle in cell population sizes that range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges. In certain embodiments, heterologous populations of immune cells or optionally the T cells therein, are contacted with a total of 1×10⁷ Cocal vesiculovirus envelope pseudotyped retroviral vector particle in cell population sizes that range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

### F. Nucleic Acids and Vectors (Nucleic Acid Vectors) Encoding the Cocal vesiculovirus Envelope Glycoprotein and Particles Containing the Glycoprotein

Codon Optimization

In some embodiments, the Cocal vesiculovirus envelope glycoprotein is encoded by a codon-optimized nucleotide sequence. In some embodiments of compositions comprising the viral particles, the viral titer and viral transduction efficiency can be increased. In some embodiments of the particle, viral transduction efficiency can be increased. Without wishing to be bound to a particular theory, it is believed that the codon-optimized nucleic acid can increase viral titer and viral transduction because the codon-optimization unexpectedly made the nucleic acid encoding the viral particle more efficient for the producer cell to express.

Accordingly, in one aspect, a nucleic acid encoding the Cocal vesiculovirus envelope protein is provided, and optionally, the nucleic acid encoding said protein has been codon-optimized.

In another aspect, a vector comprising the nucleic acid encoding the Cocal vesiculovirus envelope protein is provided, and optionally, the nucleic acid encoding said protein has been codon-optimized.

The codon-optimized nucleotide sequence can be optimized for a mammal, including a human, a rabbit, a rat, a mouse, a moose, a horse, a donkey, a guinea pig, a hamster, a monkey, a great ape, a chimpanzee, a gorilla, a bonobo, a cow, a cat, a dog, a non-human primate; a bird; a reptile; a fish; an insect, including a fruit fly; a Mollusca, and other forms of vertebrates and invertebrates including Protostomia, Deuterostomia, Chordata, Ambulacraria, Lophotrochazoa, Spiralia, Ecdysozoa, Arthropoda, Tactopoda, Panarthropoda, Gnathifera, Platytrochozoa, Rouphozoa, Gastrotricha, Platyhelminthes, Mesozoa, Annelida, Krytotrochozoa, etc. Alternatively, the codon-optimized nucleotide sequence can be optimized for a single-celled organism including a protozoa, a bacterium, and an archea. Codon optimization for humans, veterinary animals (i.e. domesticated animals), and animals used in bench-side and pre-clinical models are preferred.

In a preferred embodiment, the nucleic acid encoding the Cocal vesiculovirus envelope glycoprotein comprises: the nucleotide sequence of SEQ ID NO: 1; a nucleotide sequence with 90%-100%, 95%-100%, 96%-100%, 97%-100%, 98%-100%, 99%-100%, 90%-99%, 95%-99%, 96%-99%, 97%-99%, 98%-99%, or 99%-99.9% homology thereof; a nucleotide sequence having from 1 to 10 base pair modifications (i.e. additions, deletions, substitutions, and combinations thereof) thereof; a nucleotide sequence having from 1 to 20 base pair modifications thereof; a nucleotide sequence having from 1 to 30 base pair modifications thereof; a nucleotide sequence having from 1 to 40 base pair modifications thereof; a nucleotide sequence having from 1 to 50 base pair modifications thereof; a nucleotide sequence having at least 1, 2, 3, 4, 5, 6, 7, 8, 9, 10, 20, 25, 30, 35, 40, 45, or 50 base pair modifications thereof; or a nucleotide sequence having less than 50, 45, 40, 35, 30, 25, 20, 15, 10, 9, 8, 7, 6, 5, 4, 3, 2, or 1 modifications thereof. In some preferred embodiments the above mentioned base pair modifications and percent homology can be obtained from further codon optimization of the nucleotide sequence of SEQ ID NO:1 or can be obtained to introduce changes to the amino acid sequence encoded by SEQ ID NO:1.

In a preferred embodiment, the nucleic acid vector comprising the nucleic acid encoding the Cocal vesiculovirus envelope glycoprotein comprises: the nucleotide sequence of SEQ ID NO: 1; a nucleotide sequence with 90%-100%, 95%-100%, 96%-100%, 97%-100%, 98%-100%, 99%-100%, 90%-99%, 95%-99%, 96%-99%, 97%-99%, 98%-99%, or 99%-99.9% homology thereof; a nucleotide sequence having from 1 to 10 base pair modifications (i.e. additions, deletions, substitutions, and combinations thereof) thereof; a nucleotide sequence having from 1 to 20 base pair modifications thereof; a nucleotide sequence having from 1 to 30 base pair modifications thereof; a nucleotide sequence having from 1 to 40 base pair modifications thereof; a nucleotide sequence having from 1 to 50 base pair modifications thereof, a nucleotide sequence having at least 1, 2, 3, 4, 5, 6, 7, 8, 9, 10, 20, 25, 30, 35, 40, 45, or 50 base pair modifications thereof; or a nucleotide sequence having less than 50, 45, 40, 35, 30, 25, 20, 15, 10, 9, 8, 7, 6, 5, 4, 3, 2, or 1 modifications thereof. In preferred embodiments, the nucleic acid vector further comprises the nucleotide sequence of SEQ ID NO: 3; a nucleotide sequence with 90%-100%, 95%-100%, 96%-100%, 97%-100%, 98%-100%, 99%-100%, 90%-99%, 95%-99%, 96%-99%, 97%-99%, 98%-99%, or 99%-99.9% homology thereof; a nucleotide sequence having from 1 to 10 base pair modifications (i.e. additions, deletions, substitutions, and combinations thereof) thereof; a nucleotide sequence having from 1 to 20 base pair modifications thereof; a nucleotide sequence having from 1 to 30 base pair modifications thereof; a nucleotide sequence having from 1 to 40 base pair modifications thereof; a nucleotide sequence having from 1 to 50 base pair modifications thereof; a nucleotide sequence having at least 1, 2, 3, 4, 5, 6, 7, 8, 9, 10, 20, 25, 30, 35, 40, 45, or 50 base pair modifications thereof; or a nucleotide sequence having less than 50, 45, 40, 35, 30, 25, 20, 15, 10, 9, 8, 7, 6, 5, 4, 3, 2, or 1 modifications thereof. In some preferred embodiments the above mentioned base pair modifications and percent homology can be obtained from further codon optimization of the nucleotide sequence of SEQ ID NO:1 or can be obtained to introduce changes to the amino acid sequence encoded by SEQ ID NO:1.

In some embodiments, nucleic acid expression vectors including a nucleic acid encoding the Cocal vesiculovirus envelope protein can be introduced into a host cell used for production of the nucleic acid expression vector and by any means known to persons skilled in the art. The host cell then provides for the amplification of the nucleic acid expression vectors.

In some embodiments, nucleic acid expression vectors including a nucleic acid encoding the Cocal vesiculovirus envelope protein can be introduced into a producer cell used for production of the retroviral particle and by any means known to persons ordinarily skilled in the art. The nucleic acid expression vectors can include viral sequences for transfection, if desired. The nucleic acid expression vectors can be introduced by fusion, electroporation, biolistics, transfection, lipofection, or the like into either the producer cells or the host cells. The host and producer cells can be grown and expanded in culture before introduction of the nucleic acid vectors encoding the Cocal vesiculovirus envelope protein, followed by the appropriate treatment for introduction and integration of the nucleic acid vectors. The host and producer cells can then be expanded and screened by virtue of a reporting gene or transfection or transduction marker present in the vectors, in some embodiments. Various markers that can be used are known in the art, and can include hprt, neomycin resistance, thymidine kinase, hygromycin resistance, ampicillin resistance, green fluorescent protein, red fluorescent protein, mcherry, beta-gal, lacZ, etc. Generally, an antibiotic resistance gene can be used for determining whether the host cell has been transfected with the nucleic acid vector. Generally, a fluorescent protein can be used to determine whether a producer cell has been transfected with a nucleic acid vector. As used herein, the terms “cell,” “cell line,” and “cell culture” are used interchangeably unless otherwise indicated.

In some embodiments, the nucleic acid encoding the Cocal vesiculovirus envelope protein further comprises the nucleotide sequence of SEQ ID NO: 3, a nucleotide sequence with 90%-100%, 95%-100%, 96%-100%, 97%-100%, 98%-100%, 99%-100%, 90%-99%, 95%-99%, 96%-99%, 97%-99%, 98%-99%, or 99%-99.9% homology thereof; a nucleotide sequence having from 1 to 10 base pair modifications (including additions, deletions, or substitutions) thereof a nucleotide sequence having from 1 to 20 base pair modifications thereof; a nucleotide sequence having from 1 to 30 base pair modifications thereof; a nucleotide sequence having from 1 to 40 base pair modifications thereof a nucleotide sequence having from 1 to 50 base pair modifications thereof; a nucleotide sequence having at least 1, 2, 3, 4, 5, 6, 7, 8, 9, 10, 20, 25, 30, 35, 40, 45, or 50 base pair modifications thereof; or a nucleotide sequence having less than 50, 45, 40, 35, 30, 25, 20, 15, 10, 9, 8, 7, 6, 5, 4, 3, 2, or 1 base pair modifications thereof.

In some embodiments, the vector comprising the nucleic acid encoding the Cocal vesiculovirus envelope protein further comprises the nucleotide sequence of SEQ ID NO: 3, a nucleotide sequence with 90%-100%, 95%-100%, 96%-100%, 97%-100%, 98%-100%, 99%-100%, 90%-99%, 95%-99%, 96%-99%, 97%-99%, 98%-99%, or 99%-99.9% homology thereof; a nucleotide sequence having from 1 to 10 base pair modifications (including additions, deletions, or substitutions) thereof; a nucleotide sequence having from 1 to 20 base pair modifications thereof; a nucleotide sequence having from 1 to 30 base pair modifications thereof a nucleotide sequence having from 1 to 40 base pair modifications thereof; a nucleotide sequence having from 1 to 50 base pair modifications thereof; a nucleotide sequence having at least 1, 2, 3, 4, 5, 6, 7, 8, 9, 10, 20, 25, 30, 35, 40, 45, or 50 base pair modifications thereof; or a nucleotide sequence having less than 50, 45, 40, 35, 30, 25, 20, 15, 10, 9, 8, 7, 6, 5, 4, 3, 2, or 1 base pair modifications thereof.

In some embodiments, the nucleic acid encoding the Cocal vesiculovirus envelope protein comprises the nucleotide sequence of SEQ ID NO: 4, a nucleotide sequence with 90%-100%, 95%-100%, 96%-100%, 97%-100%, 98%-100%, 99%-100%, 90%-99%, 95%-99%, 96%-99%, 97%-99%, 98%-99%, or 99%-99.9% homology thereof; a nucleotide sequence having from 1 to 10 base pair modifications (including additions, deletions, or substitutions) thereof a nucleotide sequence having from 1 to 20 base pair modifications thereof; a nucleotide sequence having from 1 to 30 base pair modifications thereof; a nucleotide sequence having from 1 to 40 base pair modifications thereof a nucleotide sequence having from 1 to 50 base pair modifications thereof; a nucleotide sequence having at least 1, 2, 3, 4, 5, 6, 7, 8, 9, 10, 20, 25, 30, 35, 40, 45, or 50 base pair modifications thereof; or a nucleotide sequence having less than 50, 45, 40, 35, 30, 25, 20, 15, 10, 9, 8, 7, 6, 5, 4, 3, 2, or 1 base pair modifications thereof.

In some embodiments, the vector comprising the nucleic acid encoding the Cocal vesiculovirus envelope protein comprises the nucleotide sequence of SEQ ID NO: 4, a nucleotide sequence with 90%-100%, 95%-100%, 96%-100%, 97%-100%, 98%-100%, 99%-100%, 90%-99%, 95%-99%, 96%-99%, 97%-99%, 98%-99%, or 99%-99.9% homology thereof; a nucleotide sequence having from 1 to 10 base pair modifications (including additions, deletions, or substitutions) thereof a nucleotide sequence having from 1 to 20 base pair modifications thereof a nucleotide sequence having from 1 to 30 base pair modifications thereof a nucleotide sequence having from 1 to 40 base pair modifications thereof; a nucleotide sequence having from 1 to 50 base pair modifications thereof a nucleotide sequence having at least 1, 2, 3, 4, 5, 6, 7, 8, 9, 10, 20, 25, 30, 35, 40, 45, or 50 base pair modifications thereof; or a nucleotide sequence having less than 50, 45, 40, 35, 30, 25, 20, 15, 10, 9, 8, 7, 6, 5, 4, 3, 2, or 1 base pair modifications thereof.

In some embodiments, the nucleic acid encoding the Cocal vesiculovirus envelope protein consists of the nucleotide sequence of SEQ ID NO: 4, a nucleotide sequence with 90%-100%, 95%-100%, 96%-100%, 97%-100%, 98%-100%, 99%-100%, 90%-99%, 95%-99%, 96%-99%, 97%-99%, 98%-99%, or 99%-99.9% homology thereof; a nucleotide sequence having from 1 to 10 base pair modifications (including additions, deletions, or substitutions) thereof a nucleotide sequence having from 1 to 20 base pair modifications thereof; a nucleotide sequence having from 1 to 30 base pair modifications thereof; a nucleotide sequence having from 1 to 40 base pair modifications thereof a nucleotide sequence having from 1 to 50 base pair modifications thereof; a nucleotide sequence having at least 1, 2, 3, 4, 5, 6, 7, 8, 9, 10, 20, 25, 30, 35, 40, 45, or 50 base pair modifications thereof; or a nucleotide sequence having less than 50, 45, 40, 35, 30, 25, 20, 15, 10, 9, 8, 7, 6, 5, 4, 3, 2, or 1 base pair modifications thereof.

In some embodiments, the vector comprising the nucleic acid encoding the Cocal vesiculovirus envelope protein consists of the nucleotide sequence of SEQ ID NO: 4, a nucleotide sequence with 90%-100%, 95%-100%, 96%-100%, 97%-100%, 98%-100%, 99%-100%, 90%-99%, 95%-99%, 96%-99%, 97%-99%, 98%-99%, or 99%-99.9% homology thereof; a nucleotide sequence having from 1 to 10 base pair modifications (including additions, deletions, or substitutions) thereof; a nucleotide sequence having from 1 to 20 base pair modifications thereof; a nucleotide sequence having from 1 to 30 base pair modifications thereof a nucleotide sequence having from 1 to 40 base pair modifications thereof; a nucleotide sequence having from 1 to 50 base pair modifications thereof; a nucleotide sequence having at least 1, 2, 3, 4, 5, 6, 7, 8, 9, 10, 20, 25, 30, 35, 40, 45, or 50 base pair modifications thereof; or a nucleotide sequence having less than 50, 45, 40, 35, 30, 25, 20, 15, 10, 9, 8, 7, 6, 5, 4, 3, 2, or 1 base pair modifications thereof.

In the alternative, the nucleic acid encoding the Cocal vesiculovirus envelope glycoprotein can be introduced into any commercial available, any proprietary, or any newly synthesized nucleic acid vector. The following vectors are provided by way of example, and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pBacb1EG-irEG, pWLneo, pSV2cat, pOG44, PXR1, pSG (Stratagene) pSVK3, pBPV, pMSG, and pSVL (Pharmacia), pMD1, and pMD2. An expression vector can include a selectable marker, an origin of replication, restriction enzyme cleavage sites, and other features that provide for amplification, replication, manipulation, or maintenance of the vector.

In some embodiments, the nucleic acid sequence encoding the Cocal vesiculovirus envelope protein is on a separate nucleic acid or vector from nucleic acid sequences encoding other proteins, enzymes, or viral elements necessary for producing the retrovirus particles comprising or enveloped by the Cocal vesiculovirus envelope protein. In some embodiments, the nucleic acid sequence encoding the Cocal vesiculovirus envelope protein is on the same nucleic acid or vector from nucleic acid sequences encoding at least one other protein, enzyme, or viral element necessary for producing the retrovirus particles comprising or enveloped by the Cocal vesiculovirus envelope protein. In some embodiments, the nucleic acid sequence encoding the Cocal vesiculovirus envelope protein is on the same nucleic acid or vector from nucleic acid sequences encoding all the other proteins, enzymes, or viral elements necessary for producing the retrovirus particles comprising or enveloped by the Cocal vesiculovirus envelope protein.

In some embodiments, a nucleic acid of the present disclosure comprises a nucleic acid comprising one or more Cocal vesiculovirus envelope protein coding sequences or a Cocal vesiculovirus envelope protein coding sequence and one or more coding sequences of viral elements necessary for retroviral particle production is separated by a linker. A linker for use in the present disclosure allows for multiple proteins to be encoded by the same nucleic acid sequence (e.g., a multicistronic or bicistronic sequence), which are translated as a polyprotein that is dissociated into separate protein components. For example, a linker for use in a nucleic acid of the present disclosure comprising a Cocal vesiculovirus envelope protein coding sequences and coding sequences of viral elements necessary for retroviral particle production (i.e. proteins, enzymes, and elements including cis-acting and trans-acting elements or proteins including Rev, Gag/Pol, ψ, LTRs, RRE (rev response element), Env, Vif, Vpu, Vpr, and Tat) are separated by a linker, allows for the Cocal vesiculovirus envelope protein and viral elements, enzymes, and elements to be translated as a polyprotein that is dissociated into separate Cocal vesiculovirus envelope protein and retroviral proteins, enzymes, and elements (i.e. Rev).

In some embodiments, the linker comprises a nucleic acid sequence that encodes for an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunogloublin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of ordinary skill in the art would be able to select the appropriate IRES for use herein.

In some embodiments, the linker comprises a nucleic acid sequence that encodes for a self-cleaving peptide. As used herein, a “self-cleaving peptide” or “2A peptide” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A peptides are known to those of skill in the art, including, without limitation, those found in members of the Picornaviridae virus family, e.g., foot-and-mouth disease virus (FMDV), equine rhinitis A virus (ERAVO, Thosea asigna virus (TaV), and porcine tescho virus-1 (PTV-1); and carioviruses such as Theilovirus and encephalomyocarditis viruses. 2A peptides derived from FMDV, ERAV, PTV-1, and TaV are referred to herein as “F2A,” “E2A,” “P2A,” and “T2A,” respectively. Those of skill in the art would be able to select the appropriate self-cleaving peptide for use herein.

In some embodiments, a linker further comprises a nucleic acid sequence that encodes a furin cleavage site. Furin is a ubiquitously expressed protease that resides in the trans-golgi and processes protein precursors before their secretion. Furin cleaves at the COOH— terminus of its consensus recognition sequence. Various furin consensus recognition sequences (or “furin cleavage sites”) are known to those of skill in the art, including, without limitation, Arg-X1-Lys-Arg (SEQ ID NO: 29) or Arg-X1-Arg-Arg (SEQ ID NO: 30), X2-Arg-X1-X3-Arg (SEQ ID NO: 31) and Arg-X1-X1-Arg (SEQ ID NO: 32), such as an Arg-Gln-Lys-Arg (SEQ ID NO: 33), where X1 is any naturally occurring amino acid, X2 is Lys or Arg, and X3 is Lys or Arg. Those of skill in the art would be able to select the appropriate Furin cleavage site for use in the present invention.

In some embodiments, the linker comprises a nucleic acid sequence encoding a combination of a Furin cleavage site and a 2A peptide. Examples include, without limitation, a linker comprising a nucleic acid sequence encoding Furin and F2A, a linker comprising a nucleic acid sequence encoding Furin and E2A, a linker comprising a nucleic acid sequence encoding Furin and P2A, a linker comprising a nucleic acid sequence encoding Furin and T2A. Those of skill in the art would be able to select the appropriate combination for use herein. In such embodiments, the linker can further comprise a spacer sequence between the Furin and 2A peptide. Various spacer sequences are known in the art, including, without limitation, glycine serine (GS) spacers such as (GS)n, (GSGGS)n (SEQ ID NO:5) and (GGGS)n (SEQ ID NO:6), where n represents an integer of at least 1. Exemplary spacer sequences can comprise amino acid sequences including, without limitation, GGSG (SEQ ID NO:8), GGSGG (SEQ ID NO:9), GSGSG (SEQ ID NO:10), GSGGG (SEQ ID NO:11), GGGSG (SEQ ID NO:12), GSSSG (SEQ ID NO:13), and the like. Those of skill in the art would be able to select the appropriate spacer sequence.

In a certain embodiments, a vector can be structured and arranged so that the expression of the Cocal vesiculovirus envelope glycoprotein is under control of a transcriptional regulatory element. In a preferred embodiment, the vector can further comprise a transcriptional regulatory element and the transcriptional regulatory element is upstream of the Cocal vesiculovirus envelope glycoprotein (i.e. in the 5′ direction of the nucleotide sequence encoding the Cocal vesiculovirus envelope glycoprotein) and optionally, the transcriptional regulatory element controls the expression (i.e. transcription and, accordingly, but optionally, translation) of the nucleic acid encoding the Cocal vesiculovirus envelope glycoprotein. In some embodiments, the transcriptional regulatory element is constitutively active or is a constitutive promoter. In exemplary embodiments, the constitutively active transcriptional regulatory element or the constitutive promoter is a cytomegalovirus (CMV) promoter, such as the CMV major immediate early promoter (CMV IE1); a murine stem cell virus promoter; EF-1 alpha; IIRC; or SV40. In other embodiments, the activity of the transcriptional regulatory element is inducible or it is an inducible promoter. In some embodiments, the transcriptional regulatory element is a eukaryotic promoter, such as phosphoglycerate kinase promoter. Other transcriptional regulatory elements, including prokaryotic and eukaryotic, constitutive and inducible promoters, and origins of replication can be found in, for example, MOLECULAR CLONING: A LABORATORY MANUAL (Joseph F. Sambrook and David W. Russell, eds.; 3rd Ed.; Vols. 1, 2, and 3; Cold Spring Harbor Laboratory Press; 2001) and MOLECULAR CLONING: A LABORATORY MANUAL (Michael R. Green and Joseph F. Sambrook, eds.; 4th Ed.; Vols. 1, 2, and 3; Cold Spring Harbor Laboratory Press; 2012), which are incorporated by reference. These promoters are contemplated herein.

In certain embodiments, a vector can be structured and arranged so that the expression of the proteins, enzymes, and viral elements necessary for producing retroviral particles (i.e. cis-acting and trans-acting genes) are under control of a transcriptional regulatory element. In a preferred embodiment, the vector can further comprise a transcriptional regulatory element and the transcriptional regulatory element is upstream (i.e. in the 5′ direction) of the proteins, enzymes, and viral elements necessary for producing retroviral particles (i.e. cis-acting and trans-acting genes) and, optionally, the transcriptional regulatory element controls the expression (i.e. transcription or translation) of the nucleic acid encoding proteins, enzymes, and viral elements necessary for producing retroviral particles (i.e. cis-acting and trans-acting genes). In some embodiments, the transcriptional regulatory element is constitutively active or is a constitutive promoter. In exemplary embodiments, the constitutively active transcriptional regulatory element or the constitutive promoter is a cytomegalovirus (CMV) promoter, such as the CMV major immediate early promoter (CMV IE1); a murine stem cell virus promoter; EF-1 alpha; IIRC; or SV40. In other embodiments, the activity of the transcriptional regulatory element is inducible or it is an inducible promoter. In some embodiments, the transcriptional regulatory element is a eukaryotic promoter, such as phosphoglycerate kinase promoter. Other transcriptional regulatory elements, including prokaryotic and eukaryotic, constitutive and inducible promoters, can be found in, for example, MOLECULAR CLONING: A LABORATORY MANUAL (Joseph F. Sambrook and David W. Russell, eds.; 3rd Ed.; Vols. 1, 2, and 3; Cold Spring Harbor Laboratory Press; 2001) and MOLECULAR CLONING: A LABORATORY MANUAL (Michael R. Green and Joseph F. Sambrook, eds.; 4th Ed.; Vols. 1, 2, and 3; Cold Spring Harbor Laboratory Press; 2012), which are incorporated by reference. These promoters are contemplated herein.

In some embodiments, the vectors and nucleic acids encoding the Cocal vesiculovirus envelope protein must be amplified or produced prior to the introduction into producer cells and, accordingly, prior to the production of viral particles. In some embodiment, the vectors and nucleic acids encoding the other proteins, enzymes, and elements necessary for retroviral particle production must be amplified or produced prior to the introduction into producer cells, and, accordingly, the production of the retroviral proteins. In some embodiments, the vectors and nucleic acids encoding the Cocal vesiculovirus envelope protein must be structured and arranged so that a transcriptional control element drives the transcription, and therefore translation, of the Cocal vesiculovirus envelope protein in a producer cell so that the producer cell produces the retroviral particles. In some embodiments, the vectors and nucleic acids encoding the proteins, enzymes, viral elements (i.e. cis- and trans-acting genes, including rev and gag/pol) necessary for the production of the retroviral particles must be structured and arranged so that a transcriptional control element drives the transcription, and therefore translation, of the proteins, enzymes, viral elements (i.e. cis- and trans-acting genes, including rev and gag/pol) in a producer cell so that the producer cell produces the retroviral particles.

Bacterial cells, yeast cells, and animal cells can be used for amplifying or producing the nucleic acid and vectors encoding the Cocal vesiculovirus envelope protein or the proteins, enzymes, viral elements (i.e. cis- and trans-acting genes, including rev and gag/pol) necessary for the production of the retroviral particles.

For amplification in a bacterial cell, suitable promoters include, but are not limited to, lad, lacZ, T3, T7, gpt, lambda P and trc.

For amplification in a eukaryotic cell or expression in a eukaryotic cell, suitable promoters include, but are not limited to, light or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters can be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the host cell and producer cells can be from the same cell lines. In some embodiments, the host cell and the producer cell are HEK293-T cells. Accordingly, in some embodiments, the promoter can be expressed generally in all cells, or selectively in the producer cells, or specifically in the producer cells. In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

For expression in a yeast host cell for amplification, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (Lad repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences can also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, embodiments should not be limited to the use of constitutive promoters. Inducible promoters are also contemplated. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch can make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art can be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

Expression vectors generally have convenient restriction sites located near the promoter sequence to provide for the insertion of nucleic acid sequences encoding heterologous proteins (i.e. Cocal vesiculovirus envelope protein; proteins, enzymes, and elements necessary for retroviral particle production—i.e. cis- and trans-acting genes such as rev and gag/pol TCRs, and CAR). A selectable marker operative in the expression host can be present. Suitable expression vectors include, but are not limited to, retroviral vectors, whole or in part, including human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, mammary tumor virus), and the like.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

### G. Producer Cells for Making Particles Containing Cocal vesiculovirus Envelope Glycoprotein, Particles Further Containing a Transgene (i.e. a Nucleic Acid Encoding a Chimeric Antigen Receptor), and Methods for Making the Same

In another aspect, a cell is provided, the cell comprising the nucleic acid vector disclosed herein, the nucleic acid disclosed herein, or the Cocal vesiculovirus envelope protein disclosed herein. In one embodiment, the cell is a producer cell. In one embodiment, the producer cell produces a particle or viral particle containing or being enveloped by Cocal vesiculovirus envelope protein. In one embodiment, the producer cell produces a particle or viral particle containing or being enveloped by Cocal vesiculovirus envelope protein and further comprises a nucleic acid encoding a CAR. Producer cells can be generally eukaryotic cells, including immortalized cell lines and primary cell lines but can also include insect cells and insect cell lines. Immortalized cell lines can include HEK293 cells. They can further include HEK293-T cells. Other producer cells, can be found in, for example, MOLECULAR CLONING: A LABORATORY MANUAL (Joseph F. Sambrook and David W. Russell, eds.; 3rd Ed.; Vols. 1, 2, and 3; Cold Spring Harbor Laboratory Press; 2001) and MOLECULAR CLONING: A LABORATORY MANUAL (Michael R. Green and Joseph F. Sambrook, eds.; 4th Ed.; Vols. 1, 2, and 3; Cold Spring Harbor Laboratory Press; 2012), which are incorporated by reference. These producer cells are contemplated herein.

In certain embodiments, a producer cell line is generated by introducing into a cell (e.g. an immune cell) a transfer plasmid comprising a nucleotide sequence encoding a CAR, a retroviral vector comprising a nucleotide sequence encoding a Cocal vesiculovirus envelope protein, a plasmid comprising a nucleotide sequence encoding a retroviral Rev protein, and at least one plasmid comprising a nucleotide sequence encoding a retroviral Gag protein and a retroviral Pol protein. Any CAR, discussed in detail elsewhere herein, is contemplated. In certain embodiments, the amount of transfer plasmid introduced is higher than the amount of the retroviral vector comprising a nucleotide sequence encoding a Cocal vesiculovirus envelope protein. In certain embodiments, the amount of transfer plasmid introduced is at least 2 times (×), 3×, 4×, 5×, 6×, 7×, 8×, 9×, 10×, or 20× the amount of the vector comprising a nucleotide sequence encoding a Cocal vesiculovirus envelope protein. In certain embodiments, the nucleic acid sequence encoding a Cocal vesiculovirus envelope is at least 80%, at least 85%, at least 90%, at least 95%, at least 96%, at least 97%, at least 98%, at least 99%, or 100% identical to SEQ ID NO: 1. In certain embodiments, the Cocal vesiculovirus envelope protein comprises an amino acid sequence at least 80%, at least 85%, at least 90%, at least 95%, at least 96%, at least 97%, at least 98%, at least 99%, or 100% identical to SEQ ID NO: 2. In certain embodiments, the vector comprises a nucleotide sequence at least 80%, at least 85%, at least 90%, at least 95%, at least 96%, at least 97%, at least 98%, at least 99%, or 100% identical SEQ ID NO: 4.

Methods of making producer cells can include, without limitation, transforming, transfecting, or transducing the cells with a nucleic acid vector or a nucleic acid encoding the Cocal vesiculovirus envelope glycoprotein of the present disclosure along with, optionally, nucleic acid encoding the viral proteins, enzymes, and elements necessary or helpful for the producer cell to produce the viral particle. Additional methods for generating a producer cell of the present disclosure include, without limitation, chemical transformation methods (e.g., using calcium phosphate, dendrimers, liposomes, or cationic polymers), non-chemical transformation methods (e.g., electroporation, including nucleofection; optical transformation; gene electrotransfer; or hydrodynamic delivery), or particle-based methods (e.g., impalefection, using a gene gun, or magnetofection). Producer cells transfected with the nucleic acid or vector comprising the nucleic acid encoding the Cocal vesiculovirus envelope protein and other nucleic acids and vectors necessary for production of retroviral particles can then be expanded ex vivo. Producer cell lines can be transiently transfected to produce Cocal vesiculovirus vector particles. Producer cell lines can be generated to stably express all the components required for the assembly of lentivirus, which can increase vector titer and quality.

Physical methods for introducing a nucleic acid vector into a cell include calcium phosphate precipitation, lipofection, particle bombardment, microinjection, electroporation, and the like. See, e.g., Sambrook and Russell, eds., (2001), Molecular Cloning: A Laboratory Manual, Cold Spring Harbor Laboratory, New York. Chemical methods for introducing a nucleic acid vector into a cell include colloidal dispersion systems, such as macromolecule complexes, nanocapsules, microspheres, beads, and lipid-based systems including oil-in-water emulsions, micelles, mixed micelles, and liposomes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids can be obtained from Avanti Polar Lipids, Inc. (Birmingham, AL). Stock solutions of lipids in chloroform or chloroform/methanol can be stored at about −20° C. Chloroform can be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). Compositions that have different structures in solution than the normal vesicular structure are also encompassed. For example, the lipids can assume a micellar structure or merely exist as nonuniform aggregates of lipid molecules. Also contemplated are lipofectamine-nucleic acid complexes.

Regardless of the method used to introduce exogenous nucleic acids or nucleic acid vectors into the cell, a variety of assays can be performed to confirm the presence of the nucleic acids in the cell. Such assays include, for example, molecular biology assays, such as Southern and Northern blotting, reverse-transcription PCR which is optionally followed by conventional or real-time PCR; biochemistry assays, such as detecting the presence or absence of a particular peptide, e.g. the Cocal vesiculovirus envelope protein, by immunological means, e.g. ELISAs and Western blots. or by assays described herein to identify agents falling within the scope of the invention. In some embodiments, the nucleic acids or nucleic acid vectors comprise reporting genes, such as a gene encoding green fluorescent protein (GFP), that are structured and arranged to be expressed by the cell thereby causing the cell to express the reporting gene. Suitable reporter genes can include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

For example, a HEK293-T cell could fluoresce once transfected with a nucleic acid or nucleic acid vector that has a GFP reporting gene. These fluorescent HEK293-T cells could then be assessed by a fluorescent microscope, flow cytometry, or FACS, and quantified and sorted based on their level of expression of GFP. The level of expression of the reporter protein or RNA can be used as a proxy for the amount of transfection and the ability of the producer cells to produce particles and the ability of the cells to package a transgene, i.e. a nucleic acid encoding a chimeric antigen receptor, into a viral particle comprising the Cocal vesiculovirus envelope protein. For example, the level of expression of GFP, and attendant fluorescence, can be used as a proxy for the amount of transfection and the ability of the producer cells to produce particles and the ability of the cells to package a transgene, i.e. a nucleic acid encoding a chimeric antigen receptor, into a viral particle comprising the Cocal vesiculovirus envelope protein.

In some embodiments, the reporter gene is encoded on the nucleic acid vector or nucleic acid encoding the Cocal vesiculovirus envelope protein. In some embodiments, the reporter gene is encoded on the nucleic acid or nucleic acid vector encoding the cis-acting or trans-acting genes, proteins, enzymes, and viral elements necessary for production of the retroviral particle. In some embodiments, the reporter gene is encoded on the transgene that is to be encapsulated by the Cocal vesiculovirus envelope protein within the viral particle. In some embodiments, multiple reporter genes are used, such as, by way of non-limiting example, the nucleic acid vector or nucleic acid encoding the Cocal vesiculovirus envelope protein could encode a GFP reporter gene, the nucleic acid or nucleic acid vector encoding the cis-acting or trans-acting genes, proteins, enzymes, and viral elements necessary for production of the retroviral particle could further encode yellow fluorescent protein (YFP), and the transgene could encode mCherry.

In one embodiment, the nucleic acids encoding the retroviral particle introduced into the producer cell are RNA. In another embodiment, the RNA is mRNA that comprises in vitro transcribed RNA or synthetic RNA. The RNA can be produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence, or any other appropriate source of DNA.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR.

“Substantially complementary,” as used herein, refers to sequences of nucleotides where a majority or all of the bases in the primer sequence are complementary such that the nucleotide sequence is able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability or translation efficiency of the RNA can also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

In some embodiments, the RNA is electroporated into the cells, such as in vitro transcribed RNA. Any solutes suitable for cell electroporation, which can contain factors facilitating cellular permeability and viability such as sugars, peptides, lipids, proteins, antioxidants, and surfactants can be included.

In some embodiments, a nucleic acid or nucleic acid vector encoding the Cocal vesiculovirus envelope protein of the present disclosure will be RNA, e.g., in vitro synthesized RNA. Any known method can be used to synthesize RNA encoding the Cocal vesiculovirus envelope protein, the RNA encoding elements necessary and sufficient to produce the viral particles, or the RNA encoding a TCR or CAR. Methods for introducing RNA into a host cell are known in the art. See, e.g., Zhao et al. Cancer Res. (2010) 15: 9053. Introducing RNA encoding the Cocal vesiculovirus envelope protein, RNA encoding elements necessary and sufficient to produce the viral particles, and RNA encoding a transgene or nucleic acids encoding a TCR or CAR into a host cell can be carried out in vitro, ex vivo, or in vivo.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of viral proteins produced intracellularly from such a construct.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation can also be used for transfection of cells in vitro as described e.g. in US20070128708A1. Electroporation can also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

In some embodiments, the producer cells comprise helper nucleic acid vectors that are structured and arranged to produce retroviruses, including retroviruses that are self-inactivating or which lack the ability to self-propagate once they infect cells lacking the helper nucleic acid vectors cells. Accordingly, the helper nucleic acid vectors can encode and cause the expression of enzymes, proteins, and viral elements necessary and sufficient for the production of the particles or viral particles. Exemplary proteins, enzymes, and elements include cis-acting and trans-acting elements or proteins including Rev, Gag/Pol, ψ, LTRs, RRE (rev response element), Vif, Vpu, Vpr, Tat, Nef, and other envelope proteins other than Cocal vesiculovirus envelope protein. In some embodiments, an incomplete repertoire of elements are provided so that those not essential for lentiviral functions are omitted. In some embodiments, the elements or proteins therefore Gag, Pol, Tat, and Rev.

In one embodiment, one nucleic acid vector can encode the gag and pol genes, another nucleic acid vector can encode rev gene, a third can encode the envelope protein including the Cocal vesiculovirus envelope protein disclosed herein, and a fourth can encode transgenes of interest, the transgene being, for example, a CAR or a TCR.

In another aspect, the producer cell also includes a nucleic acid that encodes transgene of interest, including, for example, CAR. In some embodiments, the transgene of interest is encapsulated into the retrovirus particle when the producer cells produce the retrovirus particle. In some embodiments, the transgene of interest is a CAR. In some embodiments the CAR or TCR is encoded into the transgene of interest such that when the retrovirus particle infects a cell of interest, the cell of interest expresses (transcribes and translates) transgene, preferably the CAR or TCR, and further preferably still, the cell preferably expresses the CAR on the surface of the cell of interest. Though the retroviral vectors are able to infect a broad variety of cell types, integration and stable expression of the TCR or CAR requires the division of host cells.

In one aspect, methods of generating a Cocal vesiculovirus envelope pseudotyped retroviral vector particle are provided, the method comprising contacting a cell with one or more plasmid vectors or nucleic acid vectors comprising a nucleic acid encoding a retroviral Gag protein, a nucleic acid encoding a retroviral Pol protein, a nucleic acid encoding the Cocal vesiculovirus envelope protein. In some embodiments, the Gag or Pol proteins are from Ortervirales, including Belpaoviridae, Metaviridae, Pseudoviridae, Retroviridae (e.g. HIV), Caulimoviridae (e.g. a VII group virus family); subfamily Orthoretrovirinae, which includes genera Alpharetrovirus, Betaretrovirus, Gammaretrovirus, Deltaretrovirus, Epsilonretrovirus, Lentivirus; subfamily Spumaretrovirinae, which includes genera Bovispumavirus, Equispumavirus, Felispumavirus, Prosimiispumavirus, Simiispumavirus. Preferred embodiments include Orthoretrovirinae, Alpharetrovirus, Betaretrovirus, Deltaretrovirus, Epsilonretrovirus, Gammaretrovirus, Lentivirus, Spumaretrovirinae, Bovispumavirus, Equispumavirus, Felispumavirus, Prosimiispumavirus, and Simiispumavirus particles. In some preferred embodiments, the Gag or Pol proteins are from or derived from a retrovirus selected from a Lentivirus, an Alpharetrovirus, a Betaretrovirus, a Gammaretrovirus, a Deltaretrovirus, and an Epsilonretrovirus.

In some embodiments, the plasmid or nucleic acid vector for the expression of the Cocal vesiculovirus envelope protein comprises a nucleotide sequence that is codon-optimized, including being codon-optimized as described supra. In some embodiments, the plasmid or nucleic acid vector for the expression of the Cocal vesiculovirus envelope protein comprises a nucleotide sequence encoding the Cocal vesiculovirus envelope protein that is codon-optimized, including being codon-optimized as described supra. In some embodiments, the plasmid or nucleic acid vector for the expression of the Cocal vesiculovirus envelope protein comprises the nucleotide sequence of SEQ ID NO: 1 or SEQ ID NO: 4. In some embodiments, the plasmid or nucleic acid vector for the expression of the Cocal vesiculovirus envelope protein encodes a Cocal vesiculovirus envelope protein comprising the amino acid sequence of SEQ ID NO: 2.

### H. T Cell Receptors

In some embodiments, the particles, nucleic acid vectors, nucleic acids, and/or cells can provide compositions and methods for modifying immune cells or precursors thereof (e.g., modified T cells) so that they comprise an exogenous T cell receptor (TCR). Thus, in some embodiments, the cell has been altered to express specific T cell receptor (TCR) genes (e.g., a nucleic acid encoding an alpha/beta TCR) after transduction with the retrovirus particle comprising the Cocal vesiculovirus envelope protein. In certain embodiments, the compositions or methods can further comprise a nucleic acid that causes the knock out or knock down of the native TCR.

TCRs or antigen-binding portions thereof include those that recognize a peptide epitope or T cell epitope of a target polypeptide, such as an antigen of a tumor, viral, or autoimmune protein. In certain embodiments, the TCR redirects a T cell to a desired target. In certain embodiments, the TCR has binding specificity for a tumor associated antigen. In another embodiment, the specific TCR has higher affinity for the target cell surface antigen than a wildtype TCR.

A TCR is a disulfide-linked heterodimeric protein comprised of six different membrane bound chains that participate in the activation of T cells in response to an antigen. There exists alpha/beta TCRs and gamma/delta TCRs. An alpha/beta TCR comprises a TCR alpha chain and a TCR beta chain. T cells expressing a TCR comprising a TCR alpha chain and a TCR beta chain are commonly referred to as alpha/beta T cells. Gamma/delta TCRs comprise a TCR gamma chain and a TCR delta chain. T cells expressing a TCR comprising a TCR gamma chain and a TCR delta chain are commonly referred to as gamma/delta T cells. A TCR of the present disclosure is a TCR comprising a TCR alpha chain and a TCR beta chain.

The TCR alpha chain and the TCR beta chain are each comprised of two extracellular domains, a variable region and a constant region. The TCR alpha chain variable region and the TCR beta chain variable region are required for the affinity of a TCR to a target antigen. Each variable region comprises three hypervariable or complementarity-determining regions (CDRs) which provide for binding to a target antigen. The constant region of the TCR alpha chain and the constant region of the TCR beta chain are proximal to the cell membrane. A TCR further comprises a transmembrane region and a short cytoplasmic tail. CD3 molecules are assembled together with the TCR heterodimer. CD3 molecules comprise a characteristic sequence motif for tyrosine phosphorylation, known as immunoreceptor tyrosine-based activation motifs (ITAMs). Proximal signaling events are mediated through the CD3 molecules, and accordingly, TCR-CD3 complex interaction plays an important role in mediating cell recognition events.

Stimulation of TCR is triggered by major histocompatibility complex molecules (MHCs) on antigen presenting cells that present antigen peptides to T cells and interact with TCRs to induce a series of intracellular signaling cascades. Engagement of the TCR initiates both positive and negative signaling cascades that result in cellular proliferation, cytokine production, and/or activation-induced cell death.

A TCR can be a wild-type TCR, a high affinity TCR, and/or a chimeric TCR. A high affinity TCR can be the result of modifications to a wild-type TCR that confers a higher affinity for a target antigen compared to the wild-type TCR. A high affinity TCR can be an affinity-matured TCR. Methods for modifying TCRs and/or the affinity-maturation of TCRs are known to those of skill in the art. Techniques for engineering and expressing TCRs include, but are not limited to, the production of TCR heterodimers which include the native disulphide bridge which connects the respective subunits (Garboczi, et al., (1996), Nature 384(6605): 134-41; Garboczi, et al., (1996), J. Immunol 157(12): 5403-10; Chang et al., (1994), PNAS USA 91: 11408-11412; Davodeau et al., (1993), J. Biol. Chem. 268(21): 15455-15460; Golden et al., (1997), J. Imm. Meth. 206: 163-169; U.S. Pat. No. 6,080,840).

In some embodiments, the exogenous TCR is a full TCR or an antigen-binding portion or antigen-binding fragment thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions (CDRs) involved in recognition of the peptide, MHC, or MHC-peptide complex.

In some embodiments, the variable domains of the TCR contain hypervariable loops, or CDRs, which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al, Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen-binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen-binding and not antigen recognition (Kotb (1995) Clinical Microbiology Reviews, 8:411-426).

In some embodiments, a TCR contains a variable alpha domain (V_{α}), a variable beta domain (V_{β}), and/or antigen-binding fragments thereof. In some embodiments, the α-chain and/or β-chain of a TCR also can contain a constant domain, a transmembrane domain, and/or a short cytoplasmic tail (see, e.g., Janeway et al., Immunobiology: The Immune System in Health and Disease, 3 Ed., Current Biology Publications, p. 4:33, 1997). In some embodiments, the α chain constant domain is encoded by the TRAC gene (IMGT nomenclature) or is a variant thereof. In some embodiments, the β chain constant region is encoded by TRBC1 or TRBC2 genes (IMGT nomenclature) or is a variant thereof. In some embodiments, the constant domain is adjacent to the cell membrane. For example, in some cases, the extracellular portion of the TCR formed by the two chains contains two membrane-proximal constant domains, and two membrane-distal variable domains, which variable domains each contain CDRs.

It is within the level of a skilled artisan to determine or identify the various domains or regions of a TCR. In some aspects, residues of a TCR are known or can be identified according to the International Immunogenetics Information System (IMGT) numbering system (see e.g. www.imgt.org; see also, Lefranc et al. (2003) Developmental and Comparative Immunology, 2&;55-77; and The T Cell Factsbook 2nd Edition, Lefranc and LeFranc Academic Press 2001). Using this system, the CDR1 sequences within a TCR Va chain and/or Vβ chain correspond to the amino acids present between residue numbers 27-38, inclusive, the CDR2 sequences within a TCR Va chain and/or Vβ chain correspond to the amino acids present between residue numbers 56-65, inclusive, and the CDR3 sequences within a TCR Va chain and/or Vβ chain correspond to the amino acids present between residue numbers 105-117, inclusive. The IMGT numbering system should not be construed as limiting in any way, as there are other numbering systems known to those of skill in the art, and it is within the level of the skilled artisan to use any of the numbering systems available to identify the various domains or regions of a TCR.

In some embodiments, the TCR can be a heterodimer of two chains α and β (or optionally γ and δ) that are linked, such as by a disulfide bond or disulfide bonds. In some embodiments, the constant domain of the TCR can contain short connecting sequences in which a cysteine residue forms a disulfide bond, thereby linking the two chains of the TCR. In some embodiments, a TCR can have an additional cysteine residue in each of the α and β chains, such that the TCR contains two disulfide bonds in the constant domains. In some embodiments, each of the constant and variable domains contain disulfide bonds formed by cysteine residues.

In some embodiments, the TCR for engineering cells as described is one generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, the T-cells can be a cultured T cell hybridoma or clone. In some embodiments, the TCR or antigen-binding portion thereof can be synthetically generated from knowledge of the sequence of the TCR. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin. Cancer Res. 15: 169-180 and Cohen et al. (2005) J. Immunol. 175:5799-5808. In some embodiments, phage display is used to isolate TCRs against a target antigen (see, e.g., Varela-Rohena et al. (2008) Nat. Med. 14: 1390-1395 and Li (2005) Nat. Biotechnol. 23:349-354.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat. Immunol., 4, 55-62; Holler et al. (2000) Proc Natl. Acad. Sci. USA, 97, 5387-92), phage display (Li et al. (2005) Nat. Biotechnol., 23, 349-54), or T cell display (Chervin et al. (2008) J. Immunol. Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments as described, the TCR can contain an introduced disulfide bond or bonds. In some embodiments, the native disulfide bonds are not present. In some embodiments, the one or more of the native cysteines (e.g. in the constant domain of the α chain and β chain) that form a native interchain disulfide bond are substituted with another residue, such as with a serine or alanine. In some embodiments, an introduced disulfide bond can be formed by mutating non-cysteine residues on the alpha and beta chains, such as in the constant domain of the α chain and β chain, to cysteine. Exemplary non-native disulfide bonds of a TCR are described in published International PCT No. WO2006/000830 and WO2006/037960. In some embodiments, cysteines can be introduced at residue Thr48 of the α chain and Ser57 of the β chain, at residue Thr45 of the α chain and Ser77 of the β chain, at residue Tyr10 of the α chain and Ser17 of the β chain, at residue Thr45 of the α chain and Asp59 of the β chain and/or at residue Ser15 of the α chain and Glu15 of the β chain. In some embodiments, the presence of non-native cysteine residues (e.g. resulting in one or more non-native disulfide bonds) in a recombinant TCR can favor production of the desired recombinant TCR in a cell in which it is introduced over expression of a mismatched TCR pair containing a native TCR chain.

In some embodiments, the TCR chains contain a transmembrane domain. In some embodiments, the transmembrane domain is positively charged. In some cases, the TCR chain contains a cytoplasmic tail. In some aspects, each chain (e.g. alpha or beta) of the TCR can possess one N-terminal immunoglobulin variable domain, one immunoglobulin constant domain, a transmembrane region, and a short cytoplasmic tail at the C-terminal end. In some embodiments, a TCR, for example via the cytoplasmic tail, is associated with invariant proteins of the CD3 complex involved in mediating signal transduction. In some cases, the structure allows the TCR to associate with other molecules like CD3 and subunits thereof. For example, a TCR containing constant domains with a transmembrane region can anchor the protein in the cell membrane and associate with invariant subunits of the CD3 signaling apparatus or complex. The intracellular tails of CD3 signaling subunits (e.g. CD3y, CD35, CD3s and CD3ζ chains) contain one or more immunoreceptor tyrosine-based activation motif or ITAM that are involved in the signaling capacity of the TCR complex.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). A TCR can be cell-bound or in soluble form. In some embodiments, for purposes of the provided methods, the TCR is in cell-bound form expressed on the surface of a cell. In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native interchain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond can be desirable. In some embodiments, the TCR contains a transmembrane sequence to anchor to the membrane. In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR chain together.

In some embodiments, the TCR is a sc-TCR, which is a single amino acid strand containing an α chain and a β chain that is able to bind to MHC-peptide complexes. Typically, a scTCR can be generated using methods known to those of skill in the art, See e.g., International published PCT Nos. WO 96/13593, WO 96/18105, WO99/18129, WO04/033685, WO2006/037960, WO2011/044186; U.S. Pat. No. 7,569,664; and Schlueter, C. J. et al. J. Mol. Biol. 256, 859 (1996). In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment. In some embodiments, a scTCR contains a first segment constituted by an amino acid sequence corresponding to a TCR β chain variable region, a second segment constituted by an amino acid sequence corresponding to a TCR α chain variable region sequence fused to the N terminus of an amino acid sequence corresponding to a TCR α chain constant domain extracellular sequence, and a linker sequence linking the C terminus of the first segment to the N terminus of the second segment. In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment. In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence comprising an α chain extracellular constant domain sequence and transmembrane sequence, and, optionally, a linker sequence linking the C terminus of the first segment to the N terminus of the second segment. In some embodiments, for the scTCR to bind an WIC-peptide complex, the α and β chains must be paired so that the variable region sequences thereof are orientated for such binding. Various methods of promoting pairing of an α and β in a scTCR are well known in the art. In some embodiments, a linker sequence is included that links the a and β chains to form the single polypeptide strand. In some embodiments, the linker should have sufficient length to span the distance between the C terminus of the α chain and the N terminus of the β chain, or vice versa, while also ensuring that the linker length is not so long so that it blocks or reduces bonding of the scTCR to the target peptide-WIC complex. In some embodiments, the linker of a sc-TCRs that links the first and second TCR segments can be any linker capable of forming a single polypeptide strand, while retaining TCR binding specificity. In some embodiments, the linker sequence can, for example, have the formula -P-AA-P-, wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, a scTCR contains a disulfide bond between residues of the single amino acid strand, which, in some cases, can promote stability of the pairing between the α and β regions of the single chain molecule (see e.g. U.S. Pat. No. 7,569,664). In some embodiments, the scTCR contains a covalent disulfide bond linking a residue of the immunoglobulin region of the constant domain of the α chain to a residue of the immunoglobulin region of the constant domain of the R chain of the single chain molecule. In some embodiments, the disulfide bond corresponds to the native disulfide bond present in a native dTCR. In some embodiments, the disulfide bond in a native TCR is not present. In some embodiments, the disulfide bond is an introduced non-native disulfide bond, for example, by incorporating one or more cysteines into the constant region extracellular sequences of the first and second chain regions of the scTCR polypeptide. Exemplary cysteine mutations include any as described above. In some cases, both a native and a non-native disulfide bond can be present.

In some embodiments, any of the TCRs, including a dTCR or scTCR, can be linked to signaling domains that yield an active TCR on the surface of a T cell. In some embodiments, the TCR is expressed on the surface of cells. In some embodiments, the TCR does contain a sequence corresponding to a transmembrane sequence. In some embodiments, the transmembrane domain can be a Ca or CP transmembrane domain. In some embodiments, the transmembrane domain can be from a non-TCR origin, for example, a transmembrane region from CD3z, CD28 or B7.1. In some embodiments, the TCR does contain a sequence corresponding to cytoplasmic sequences. In some embodiments, the TCR contains a CD3z signaling domain. In some embodiments, the TCR is capable of forming a TCR complex with CD3. In some embodiments, the TCR or antigen-binding portion thereof can be a recombinantly produced natural protein or mutated form thereof in which one or more property, such as binding characteristic, has been altered. In some embodiments, a TCR can be derived from one of various animal species, such as human, mouse, rat, or other mammal.

In some embodiments, the TCR comprises affinity to a target antigen on an antigen-target cell. The target antigen can include any type of protein, or epitope thereof, associated with the antigen-target cell. For example, the TCR can comprise affinity to a target antigen on a target cell that indicates a particular disease state of the target cell. In some embodiments, the target antigen is processed and presented by MHCs.

### I. Methods of Producing Genetically Modified Immune Cells

The present disclosure provides methods for producing or generating a modified immune cell or precursor thereof (e.g., a T cell) for tumor immunotherapy, e.g., adoptive immunotherapy. The cells generally are engineered by introducing one or more genetically engineered nucleic acids encoding the exogenous receptors (e.g., a TCR and/or CAR) by infecting the target cell with a Cocal vesiculovirus enveloped pseudotyped retroviral vector particle comprising a transgene encoding the exogenous receptor.

In certain embodiments, the nucleic acid encoding an exogenous TCR and/or CAR is introduced into the cell via retroviral transduction. In certain embodiments, the viral transduction comprises contacting the immune or precursor cell with a retroviral particle or retroviral vector comprising the nucleic acid encoding an exogenous TCR and/or CAR.

Retrovirus expression vectors are capable of integrating into the host genome, delivering a large amount of foreign genetic material, infecting a broad spectrum of species and cell types and being packaged in special cell lines. In some embodiments, the retroviral vector is constructed by inserting a nucleic acid (e.g., a nucleic acid encoding an exogenous TCR and/or CAR) into the retroviral genome at certain locations to produce a retrovirus that optionally is replication defective or self-inactivating. Though the retroviral vectors are able to infect a broad variety of cell types, integration and stable expression of the TCR and/or CAR can require the division of host cells.

The method of transducing a cell with the retroviruses comprising or encapsulated by the Cocal vesiculovirus envelope protein disclosed herein is not limited to any one subfamily, genera, or pseudotype of lentivirus, and as noted supra, the retrovirus can include elements of multiple subfamilies, genera, and pseudotypes of retrovirus. In some embodiments, lentiviruses are preferred, including Human Immunodeficiency Viruses (HIV-1, HIV-2) and Simian Immunodeficiency Viruses (SIV). Lentiviral vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Lentiviral vectors have been generated by multiply attenuating the HIV virulence genes, for example, the genes env, vif, vpr, vpu, and nef are deleted making the vector biologically safe. Lentiviral vectors are capable of infecting non-dividing cells and can be used for both in vivo and ex vivo gene transfer and expression, e.g., of a nucleic acid encoding a TCR or CAR (see, e.g., U.S. Pat. No. 5,994,136).

In some embodiments, the method of delivering a nucleic acid sequence encoding a TCR or CAR by contacting the immune cell with a Cocal vesiculovirus enveloped pseudotyped retroviral vector particle results in genetically engineered cells including genetically engineered T-lymphocytes (T cells), naive T cells (TN), memory T cells (for example, central memory T cells (TCM), effector memory cells (TEM)), natural killer cells (NK cells), and macrophages capable of giving rise to therapeutically relevant progeny. In certain embodiments, the genetically engineered cells are autologous cells. In certain embodiments, the modified cell is resistant to T cell exhaustion.

In some embodiments, the immune cells (e.g. T cells) can be incubated or cultivated prior to, during, or subsequent to introducing particles or compositions comprising the particles containing the nucleic acid encoding the CAR or TCR and the Cocal vesiculovirus envelope protein. In some embodiments, the cells (e.g. T cells) can be incubated or cultivated prior to, during or subsequent to the contacting, such as prior to, during or subsequent to the transduction of the cells with a viral vector (e.g. lentiviral vector) encoding the exogenous receptor. In some embodiments, the method includes activating or stimulating cells with a stimulating or activating agent (e.g. anti-CD3/anti-CD28 antibodies) prior to the contacting with the particles. In some embodiments, prior to the introducing of the agent, the cells are allowed to rest, e.g. by removal of any stimulating or activating agent. In some embodiments, prior to introducing the agent, the stimulating or activating agent and/or cytokines are not removed.

### J. Nucleic Acids and Expression Vectors Encapsulated in the Cocal vesiculovirus Envelope Pseudotyped Retroviral Vector Particle and Encoding the TCR and/or CAR

While the present disclosure provides for nucleic acids and vectors encoding the Cocal vesiculovirus envelope protein, retroviral particles comprising and encapsulating said proteins, and cells producing said viral particles, the present disclosure also provides for nucleic acids and vectors encapsulated by said particles, these nucleic acids and vectors encoding a transgene that can be transduced into a cell infected by the retroviral particle. Accordingly, the present disclosure provides a nucleic acid and vectors encoding an exogenous TCR or CAR, the nucleic acids and vectors being capable of being packaged within the retroviral particle. In one embodiment, a nucleic acid of the present disclosure comprises a nucleic acid sequence encoding an exogenous TCR. In one embodiment, a nucleic acid of the present disclosure comprises a nucleic acid sequence encoding an exogenous CAR.

In some embodiments, a nucleic acid or vector of the present disclosure is provided for the production of a TCR and/or CAR as described herein, e.g., in a mammalian cell. In some embodiments, a nucleic acid or vector of the present disclosure provides for amplification of the TCR- or CAR-encoding nucleic acid.

As described herein, a TCR of the present disclosure comprises a TCR alpha chain and a TCR beta chain. Accordingly, the present disclosure provides a nucleic acid encoding a TCR alpha chain, and a nucleic acid encoding a TCR beta chain. In some embodiments, the nucleic acid encoding a TCR alpha chain is separate from the nucleic acid encoding a TCR beta chain. In an exemplary embodiment, the nucleic acid encoding a TCR alpha chain, and the nucleic acid encoding a TCR beta chain, resides within the same nucleic acid.

In some embodiments, a nucleic acid of the present disclosure comprises a nucleic acid comprising a TCR alpha chain coding sequence and a TCR beta chain coding sequence. In some embodiments, a nucleic acid of the present disclosure comprises a nucleic acid comprising a TCR alpha chain coding sequence and a TCR beta chain coding sequence that is separated by a linker. A linker for use in the present disclosure allows for multiple proteins to be encoded by the same nucleic acid sequence (e.g., a multicistronic or bicistronic sequence), which are translated as a polyprotein that is dissociated into separate protein components. For example, a linker for use in a nucleic acid of the present disclosure comprising a TCR alpha chain coding sequence and a TCR beta chain coding sequence, allows for the TCR alpha chain and TCR beta chain to be translated as a polyprotein that is dissociated into separate TCR alpha chain and TCR beta chain components.

In some embodiments, the linker comprises a nucleic acid sequence that encodes for an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunogloublin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES for use in herein.

In some embodiments, the linker comprises a nucleic acid sequence that encodes for a self-cleaving peptide. As used herein, a “self-cleaving peptide” or “2A peptide” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A peptides are known to those of skill in the art, including, without limitation, those found in members of the Picornaviridae virus family, e.g., foot-and-mouth disease virus (FMDV), equine rhinitis A virus (ERAVO, Thosea asigna virus (TaV), and porcine tescho virus-1 (PTV-1); and carioviruses such as Theilovirus and encephalomyocarditis viruses. 2A peptides derived from FMDV, ERAV, PTV-1, and TaV are referred to herein as “F2A,” “E2A,” “P2A,” and “T2A,” respectively. Those of skill in the art would be able to select the appropriate self-cleaving peptide for use herein.

In some embodiments, a linker further comprises a nucleic acid sequence that encodes a furin cleavage site. Furin is a ubiquitously expressed protease that resides in the trans-golgi and processes protein precursors before their secretion. Furin cleaves at the COOH— terminus of its consensus recognition sequence. Various furin consensus recognition sequences (or “furin cleavage sites”) are known to those of skill in the art.

In some embodiments, the linker comprises a nucleic acid sequence encoding a combination of a Furin cleavage site and a 2A peptide. Examples include, without limitation, a linker comprising a nucleic acid sequence encoding Furin and F2A, a linker comprising a nucleic acid sequence encoding Furin and E2A, a linker comprising a nucleic acid sequence encoding Furin and P2A, a linker comprising a nucleic acid sequence encoding Furin and T2A. Those of skill in the art would be able to select the appropriate combination for use herein. In such embodiments, the linker can further comprise a spacer sequence between the Furin and 2A peptide. Various spacer sequences are known in the art.

In some embodiments, a nucleic acid of the present disclosure can be operably linked to a transcriptional control element, e.g., a promoter, and enhancer, etc. Suitable promoter and enhancer elements are known to those of skill in the art.

In certain embodiments, the nucleic acid encoding an exogenous TCR and/or CAR is in operable linkage with a promoter. In certain embodiments, the promoter is a phosphoglycerate kinase-1 (PGK) promoter.

It can be necessary to produce the vector encoding the CAR or TCR prior to introduction into the producer cell, and prior to encapsulation in the Cocal vesiculovirus envelope protein. Accordingly, it can be necessary to amplify or express the vector encoding the exogenous TCR or CAR in a host cell prior to introduction into a producer cell. For expression in a bacterial cell, suitable promoters include, but are not limited to, lad, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters can be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (Lad repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences can also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the embodiments are not be limited to the use of constitutive promoters. Inducible promoters are also contemplated. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch can make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art can be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

A nucleic acid encoding a CAR or a TCR can be present within an expression vector and/or a cloning vector. An expression vector can include a selectable marker, an origin of replication, and other features that provide for replication, modification of, or maintenance of the vector. Suitable expression vectors include, e.g., plasmids, viral vectors, and the like. Large numbers of suitable vectors and promoters are known to those of skill in the art; many are commercially available for generating a subject recombinant construct. Suitable expression vectors include retroviral vectors, whole or in part, including human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, mammary tumor virus), and the like. Additional expression vectors suitable for use are, e.g., without limitation, a lentivirus vector, a gamma retrovirus vector, a foamy virus vector, and an engineered hybrid virus vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals.

Expression vectors generally have convenient restriction sites located near the promoter sequence to provide for the insertion of nucleic acid sequences encoding heterologous proteins. A selectable marker operative in the expression host can be present.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, the Cocal vesiculovirus enveloped pseudotyped retroviral vector particle can be used to introduce the TCR or CAR into an immune cell or precursor thereof (e.g., a T cell). In some embodiments, the Cocal vesiculovirus enveloped pseudotyped retroviral vector particle will comprise additional elements that will aid in the functional expression of the TCR or CAR encoded therein. In some embodiments, an expression vector comprising a nucleic acid encoding for a TCR or CAR further comprises a mammalian promoter. In one embodiment, the vector further comprises an elongation-factor-1-alpha promoter (EF-1α promoter). Use of an EF-1α promoter can increase the efficiency in expression of downstream transgenes (e.g., a TCR and/or CAR encoding nucleic acid sequence). Physiologic promoters (e.g., an EF-1α promoter) can be less likely to induce integration mediated genotoxicity, and can abrogate the ability of the retroviral vector to transform stem cells. Other physiological promoters suitable for use in a Cocal vesiculovirus enveloped pseudotyped retroviral vector particle are known to those of skill in the art and can be incorporated into exemplary embodiments of the nucleic acid vector.

In some embodiments, Cocal vesiculovirus enveloped pseudotyped retroviral vector particle further comprises a non-requisite cis-acting sequence that can improve titers and gene expression. One non-limiting example of a non-requisite cis-acting sequence is the central polypurine tract and central termination sequence (cPPT/CTS) which is important for efficient reverse transcription and nuclear import. Other non-requisite cis-acting sequences are known to those of skill in the art and can be incorporated into a Cocal vesiculovirus enveloped pseudotyped retroviral vector particle. In some embodiments, the nucleic acid vector encoding the CAR or TCR further comprises a posttranscriptional regulatory element. Posttranscriptional regulatory elements can improve RNA translation, improve transgene expression and stabilize RNA transcripts. One example of a posttranscriptional regulatory element is the woodchuck hepatitis virus posttranscriptional regulatory element (WPRE). Accordingly, in some embodiments a nucleic acid vector further comprises a WPRE sequence. Various posttranscriptional regulator elements are known to those of skill in the art and can be incorporated into Cocal vesiculovirus enveloped pseudotyped retroviral vector particle or nucleic acid vector capable of being contained therein. A vector can further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, the Cocal vesiculovirus enveloped pseudotyped retroviral vector particle and nucleic acid vector capable of being contained therein includes a 3′ U3 deleted LTR. Accordingly, Cocal vesiculovirus enveloped pseudotyped retroviral vector particle or nucleic acid vector capable of being contained therein can comprise any combination of the elements described herein to enhance the efficiency of functional expression of transgenes. For example, a Cocal vesiculovirus enveloped pseudotyped retroviral vector particle or nucleic acid capable of being contained therein can comprise a WPRE sequence, cPPT sequence, RRE sequence, 5′LTR, 3′ U3 deleted LTR′ in addition to a nucleic acid encoding for a TCR or CAR.

Vectors of exemplary embodiments can be self-inactivating vectors. As used herein, the term “self-inactivating vector” refers to vectors in which the 3′ LTR enhancer promoter region (U3 region) has been modified (e.g., by deletion or substitution). A self-inactivating vector can prevent viral transcription beyond the first round of viral replication. Consequently, a self-inactivating vector can be capable of infecting and then integrating into a host genome (e.g., a mammalian genome) only once, and cannot be passed further. Accordingly, self-inactivating vectors can greatly reduce the risk of creating a replication-competent virus.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes can be made and equivalents can be substituted without departing from the true spirit and scope of the invention. It will be readily apparent to those skilled in the art that other suitable modifications and adaptations of the methods described herein can be made using suitable equivalents without departing from the scope of the embodiments disclosed herein. In addition, many modifications can be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

TABLE 1
Amino acid and nucleotide sequences

| | SEQ | |
|---|---|---|
| Name: | ID NO: | Sequence |
| Cocal | 1 | atgaacttcctcctgctgacttttatcgtgctgcctctctgctcccacgccaagttctcgattgtgttccccc |
| Envelope | | aatcccaaaaggggaactggaagaatgtgccctcctcgtaccactactgcccgtcctcctccgaccaa |
| Nucleic | | aactggcacaacgatctgctcggaatcaccatgaaggtcaagatgcccaagacccataaggctattca |
| Acid | | ggccgacggctggatgtgccacgccgcgaagtggatcaccacctgtgacttccggtggtacggtccg |
| | | aagtacatcactcactcgattcactcaattcagccgactagcgagcagtgcaaagagagcatcaagca |
| | | gacgaagcagggcacatggatgtcccccggattccctccccaaaactgcggatatgcgaccgtgacc |
| | | gatagcgtggccgtggtggtgcaggccacccctcatcatgtgcttgtggatgagtacaccggagaatg |
| | | gatcgacagccagttcccgaacggaaaatgcgaaaccgaggagtgcgagactgtccacaactccac |
| | | tgtgtggtactccgactacaaggtcacgggcttgtgcgacgcgactttggtggacaccgaaatcacctt |
| | | ctttagcgaggatggaaagaaggagtccatcggcaaaccgaacactggttaccgctccaattacttcg |
| | | cgtacgaaaagggagacaaagtctgcaagatgaattactgcaagcacgccggtgtcaggctgccatc |
| | | aggagtgtggttcgaattcgtggaccaggacgtgtacgctgccgcgaagcttccggaatgtccagtcg |
| | | gggcaaccatttccgcaccgactcagacctctgtggatgtgtccctgatcctggacgtcgagagaatc |
| | | ctggactacagcctgtgtcaggagacttggtcgaagattcgctccaagcagcccgtgtcacctgtggat |
| | | ctgtcgtatctggccccgaagaaccctggtaccggcccagcctttaccatcataaacgggaccctgaa |
| | | gtacttcgaaactcggtatattcggattgacatcgacaaccccatcatctcgaaaatggtcggaaagatc |
| | | agcggatcccagacagaaagggaactctggaccgaatggttcccgtacgagggcgtggaaatcggt |
| | | ccgaacgggatcctgaaaactcctacgggctacaagttccccctcttcatgattgggcatggcatgctg |
| | | gactccgatctccacaagacctcccaagctgaagtgttcgagcaccctcacctggccgaagcaccca |
| | | agcagctgccagaggaagaaaccctcttcttcggggacaccggaatctcgaagaacccggtggaac |
| | | tgattgagggctggttctcatcatggaagtccaccgtggtcaccttcttcttcgccatcggagtgtttatcc |
| | | tgctttacgtggtggcccgcatcgtgattgccgtgcggtacagataccagggctccaacaacaagcgc |
| | | atctacaacgatatcgagatgagccggttccgcaagtaa |
| Cocal | 2 | MNFLLLTFIVLPLCSHAKFSIVFPQSQKGNWKNVPSSYHYCPSSS |
| Envelope | | DQNWHNDLLGITMKVKMPKTHKAIQADGWMCHAAKWITTCD |
| Amino | | FRWYGPKYITHSIHSIQPTSEQCKESIKQTKQGTWMSPGFPPQNC |
| Acid | | GYATVTDSVAVVVQATPHHVLVDEYTGEWIDSQFPNGKCETEE |
| | | CETVHNSTVWYSDYKVTGLCDATLVDTEITFFSEDGKKESIGKP |
| | | NTGYRSNYFAYEKGDKVCKMNYCKHAGVRLPSGVWFEFVDQD |
| | | VYAAAKLPECPVGATISAPTQTSVDVSLILDVERILDYSLCQETW |
| | | SKIRSKQPVSPVDLSYLAPKNPGTGPAFTIINGTLKYFETRYIRIDI |
| | | DNPIISKMVGKISGSQTERELWTEWFPYEGVEIGPNGILKTPTGY |
| | | KFPLFMIGHGMLDSDLHKTSQAEVFEHPHLAEAPKQLPEEETLFF |
| | | GDTGISKNPVELIEGWFSSWKSTVVTFFFAIGVFILLYVVARIVIA |
| | | VRYRYQGSNNKRIYNDIEMSRFRK |
| Vector | 3 | ggatcccctgagggggcccccatgggctagaggatccggcctcggcctctgcataaataaaaaaaat |
| | | tagtcagccatgagcttggcccattgcatacgttgtatccatatcataatatgtacatttatattggctcatgt |
| | | ccaacattaccgccatgttgacattgattattgactagttattaatagtaatcaattacggggtcattagttc |
| | | atagcccatatatggagttccgcgttacataacttacggtaaatggcccgcctggctgaccgcccaacg |
| | | acccccgcccattgacgtcaataatgacgtatgttcccatagtaacgccaatagggactttccattgacg |
| | | tcaatgggtggagtatttacggtaaactgcccacttggcagtacatcaagtgtatcatatgccaagtacg |
| | | ccccctattgacgtcaatgacggtaaatggcccgcctggcattatgcccagtacatgaccttatgggac |
| | | tttcctacttggcagtacatctacgtattagtcatcgctattaccatggtgatgcggttttggcagtacatca |
| | | atgggcgtggatagcggtttgactcacggggatttccaagtctccaccccattgacgtcaatgggagttt |
| | | gttttggcaccaaaatcaacgggactttccaaaatgtcgtaacaactccgccccattgacgcaaatggg |
| | | cggtaggcgtgtacggtgggaggtctatataagcagagctcgtttagtgaaccgtcagatcgcctgga |
| | | gacgccatccacgctgttttgacctccatagaagacaccgggaccgatccagcctcccctcgaagctt |
| | | acatgtggtaccgagctcggatcctgagaacttcagggtgagtctatgggacccttgatgttttctttccc |
| | | cttcttttctatggttaagttcatgtcataggaaggggagaagtaacagggtacacatattgaccaaatca |
| | | gggtaattttgcatttgtaattttaaaaaatgctttcttcttttaatatacttttttgtttatcttatttctaatactttc |
| | | cctaatctctttctttcagggcaataatgatacaatgtatcatgcctctttgcaccattctaaagaataacag |
| | | tgataatttctgggttaaggcaatagcaatatttctgcatataaatatttctgcatataaattgtaactgatgt |
| | | aagaggtttcatattgctaatagcagctacaatccagctaccattctgcttttattttatggttgggataagg |
| | | ctggattattctgagtccaagctaggcccttttgctaatcatgttcatacctcttatcttcctcccacagctcc |
| | | tgggcaacgtgctggtctgtgtgctggcccatcactttggcaaagcacgtgagatctgaattctgacact |
| | | ctcaaatcctgcacaacagattcttcatgtttggaccaaatcaacttgtgataccatgctcaaagaggcct |
| | | caattatatttgagtttttaatttttatgaaaaaaaaaaaaaaaaacggaattcaccccaccagtgcaggct |
| | | gcctatcagaaagtggtggctggtgtggctaatgccctggcccacaagtatcactaagctcgctttcttg |
| | | ctgtccaatttctattaaaggttcctttgttccctaagtccaactactaaactgggggatattatgaagggc |
| | | cttgagcatctggattctgcctaataaaaaacatttattttcattgcaatgatgtatttaaattatttctgaatat |
| | | tttactaaaaagggaatgtgggaggtcagtgcatttaaaacataaagaaatgaagagctagttcaaacc |
| | | ttgggaaaatacactatatcttaaactccatgaaagaaggtgaggctgcaaacagctaatgcacattgg |
| | | caacagcccctgatgcctatgccttattcatccctcagaaaaggattcaagtagaggcttgatttggagg |
| | | ttaaagttttgctatgctgtattttacattacttattgttttagctgtcctcatgaatgtcttttcactacccatttg |
| | | cttatcctgcatctctcagccttgactccactcagttctcttgcttagagataccacctttcccctgaagtgtt |
| | | ccttccatgttttacggcgagatggtttctcctcgcctggccactcagccttagttgtctctgttgtcttatag |
| | | aggtctacttgaagaaggaaaaacagggggcatggtttgactgtcctgtgagcccttcttccctgcctc |
| | | ccccactcacagtgacccggaatccctcgacatggcagtctagcactagtgcggccgcagatctgctt |
| | | cctcgctcactgactcgctgcgctcggtcgttcggctgcggcgagcggtatcagctcactcaaaggcg |
| | | gtaatacggttatccacagaatcaggggataacgcaggaaagaacatgtgagcaaaaggccagcaa |
| | | aaggccaggaaccgtaaaaaggccgcgttgctggcgtttttccataggctccgcccccctgacgagc |
| | | atcacaaaaatcgacgctcaagtcagaggtggcgaaacccgacaggactataaagataccaggcgtt |
| | | tccccctggaagctccctcgtgcgctctcctgttccgaccctgccgcttaccggatacctgtccgccttt |
| | | ctcccttcgggaagcgtggcgctttctcatagctcacgctgtaggtatctcagttcggtgtaggtcgttcg |
| | | ctccaagctgggctgtgtgcacgaaccccccgttcagcccgaccgctgcgccttatccggtaactatc |
| | | gtcttgagtccaacccggtaagacacgacttatcgccactggcagcagccactggtaacaggattagc |
| | | agagcgaggtatgtaggcggtgctacagagttcttgaagtggtggcctaactacggctacactagaag |
| | | aacagtatttggtatctgcgctctgctgaagccagttaccttcggaaaaagagttggtagctcttgatccg |
| | | gcaaacaaaccaccgctggtagcggtggtttttttgtttgcaagcagcagattacgcgcagaaaaaaa |
| | | ggatctcaagaagatcctttgatcttttctacggggtctgacgctcagtggaacgaaaactcacgttaag |
| | | ggattttggtcatgagattatcaaaaaggatcttcacctagatccttttaaattaaaaatgaagttttaaatc |
| | | aatctaaagtatatatgagtaaacttggtctgacagttaccaatgcttaatcagtgaggcacctatctcag |
| | | cgatctgtctatttcgttcatccatagttgcctgactccccgtcgtgtagataactacgatacgggagggc |
| | | ttaccatctggccccagtgctgcaatgataccgcgagacccacgctcaccggctccagatttatcagca |
| | | ataaaccagccagccggaagggccgagcgcagaagtggtcctgcaactttatccgcctccatccagt |
| | | ctattaattgttgccgggaagctagagtaagtagttcgccagttaatagtttgcgcaacgttgttgccattg |
| | | ctacaggcatcgtggtgtcacgctcgtcgtttggtatggcttcattcagctccggttcccaacgatcaag |
| | | gcgagttacatgatcccccatgttgtgcaaaaaagcggttagctccttcggtcctccgatcgttgtcaga |
| | | agtaagttggccgcagtgttatcactcatggttatggcagcactgcataattctcttactgtcatgccatcc |
| | | gtaagatgcttttctgtgactggtgagtactcaaccaagtcattctgagaatagtgtatgcggcgaccga |
| | | gttgctcttgcccggcgtcaatacgggataataccgcgccacatagcagaactttaaaagtgctcatcat |
| | | tggaaaacgttcttcggggcgaaaactctcaaggatcttaccgctgttgagatccagttcgatgtaaccc |
| | | actcgtgcacccaactgatcttcagcatcttttactttcaccagcgtttctgggtgagcaaaaacaggaa |
| | | ggcaaaatgccgcaaaaaagggaataagggcgacacggaaatgttgaatactcatactcttcctttttc |
| | | aatattattgaagcatttatcagggttattgtctcatgagcggatacatatttgaatgtatttagaaaaataaa |
| | | caaataggggttccgcgcacatttccccgaaaagtgccacctgacgt |
| Vector | 4 | ggatcccctgagggggcccccatgggctagaggatccggcctcggcctctgcataaataaaaaaaat |
| Contaning | | tagtcagccatgagcttggcccattgcatacgttgtatccatatcataatatgtacatttatattggctcatgt |
| Cocal | | ccaacattaccgccatgttgacattgattattgactagttattaatagtaatcaattacggggtcattagttc |
| Envelope | | atagcccatatatggagttccgcgttacataacttacggtaaatggcccgcctggctgaccgcccaacg |
| | | acccccgcccattgacgtcaataatgacgtatgttcccatagtaacgccaatagggactttccattgacg |
| | | tcaatgggtggagtatttacggtaaactgcccacttggcagtacatcaagtgtatcatatgccaagtacg |
| | | ccccctattgacgtcaatgacggtaaatggcccgcctggcattatgcccagtacatgaccttatgggac |
| | | tttcctacttggcagtacatctacgtattagtcatcgctattaccatggtgatgcggttttggcagtacatca |
| | | atgggcgtggatagcggtttgactcacggggatttccaagtctccaccccattgacgtcaatgggagttt |
| | | gttttggcaccaaaatcaacgggactttccaaaatgtcgtaacaactccgccccattgacgcaaatggg |
| | | cggtaggcgtgtacggtgggaggtctatataagcagagctcgtttagtgaaccgtcagatcgcctgga |
| | | gacgccatccacgctgttttgacctccatagaagacaccgggaccgatccagcctcccctcgaagctt |
| | | acatgtggtaccgagctcggatcctgagaacttcagggtgagtctatgggacccttgatgttttctttccc |
| | | cttcttttctatggttaagttcatgtcataggaaggggagaagtaacagggtacacatattgaccaaatca |
| | | gggtaattttgcatttgtaattttaaaaaatgctttcttcttttaatatacttttttgtttatcttatttctaatactttc |
| | | cctaatctctttctttcagggcaataatgatacaatgtatcatgcctctttgcaccattctaaagaataacag |
| | | tgataatttctgggttaaggcaatagcaatatttctgcatataaatatttctgcatataaattgtaactgatgt |
| | | aagaggtttcatattgctaatagcagctacaatccagctaccattctgcttttattttatggttgggataagg |
| | | ctggattattctgagtccaagctaggcccttttgctaatcatgttcatacctcttatcttcctcccacagctcc |
| | | tgggcaacgtgctggtctgtgtgctggcccatcactttggcaaagcacgtgagatctgaattctgacact |
| | | atgaacttcctcctgctgacttttatcgtgctgcctctctgctcccacgccaagttctcgattgtgttccccc |
| | | aatcccaaaaggggaactggaagaatgtgccctcctcgtaccactactgcccgtcctcctccgaccaa |
| | | aactggcacaacgatctgctcggaatcaccatgaaggtcaagatgcccaagacccataaggctattca |
| | | ggccgacggctggatgtgccacgccgcgaagtggatcaccacctgtgacttccggtggtacggtccg |
| | | aagtacatcactcactcgattcactcaattcagccgactagcgagcagtgcaaagagagcatcaagca |
| | | gacgaagcagggcacatggatgtcccccggattccctccccaaaactgcggatatgcgaccgtgacc |
| | | gatagcgtggccgtggtggtgcaggccacccctcatcatgtgcttgtggatgagtacaccggagaatg |
| | | gatcgacagccagttcccgaacggaaaatgcgaaaccgaggagtgcgagactgtccacaactccac |
| | | tgtgtggtactccgactacaaggtcacgggcttgtgcgacgcgactttggtggacaccgaaatcacctt |
| | | ctttagcgaggatggaaagaaggagtccatcggcaaaccgaacactggttaccgctccaattacttcg |
| | | cgtacgaaaagggagacaaagtctgcaagatgaattactgcaagcacgccggtgtcaggctgccatc |
| | | aggagtgtggttcgaattcgtggaccaggacgtgtacgctgccgcgaagcttccggaatgtccagtcg |
| | | gggcaaccatttccgcaccgactcagacctctgtggatgtgtccctgatcctggacgtcgagagaatc |
| | | ctggactacagcctgtgtcaggagacttggtcgaagattcgctccaagcagcccgtgtcacctgtggat |
| | | ctgtcgtatctggccccgaagaaccctggtaccggcccagcctttaccatcataaacgggaccctgaa |
| | | gtacttcgaaactcggtatattcggattgacatcgacaaccccatcatctcgaaaatggtcggaaagatc |
| | | agcggatcccagacagaaagggaactctggaccgaatggttcccgtacgagggcgtggaaatcggt |
| | | ccgaacgggatcctgaaaactcctacgggctacaagttccccctcttcatgattgggcatggcatgctg |
| | | gactccgatctccacaagacctcccaagctgaagtgttcgagcaccctcacctggccgaagcaccca |
| | | agcagctgccagaggaagaaaccctcttcttcggggacaccggaatctcgaagaacccggtggaac |
| | | tgattgagggctggttctcatcatggaagtccaccgtggtcaccttcttcttcgccatcggagtgtttatcc |
| | | tgctttacgtggtggcccgcatcgtgattgccgtgcggtacagataccagggctccaacaacaagcgc |
| | | atctacaacgatatcgagatgagccggttccgcaagtaactcaaatcctgcacaacagattcttcatgttt |
| | | ggaccaaatcaacttgtgataccatgctcaaagaggcctcaattatatttgagtttttaatttttatgaaaaa |
| | | aaaaaaaaaaaacggaattcaccccaccagtgcaggctgcctatcagaaagtggtggctggtgtggc |
| | | taatgccctggcccacaagtatcactaagctcgctttcttgctgtccaatttctattaaaggttcctttgttcc |
| | | ctaagtccaactactaaactgggggatattatgaagggccttgagcatctggattctgcctaataaaaaa |
| | | catttattttcattgcaatgatgtatttaaattatttctgaatattttactaaaaagggaatgtgggaggtcagt |
| | | gcatttaaaacataaagaaatgaagagctagttcaaaccttgggaaaatacactatatcttaaactccatg |
| | | aaagaaggtgaggctgcaaacagctaatgcacattggcaacagcccctgatgcctatgccttattcatc |
| | | cctcagaaaaggattcaagtagaggcttgatttggaggttaaagttttgctatgctgtattttacattacttat |
| | | tgttttagctgtcctcatgaatgtcttttcactacccatttgcttatcctgcatctctcagccttgactccactc |
| | | agttctcttgcttagagataccacctttcccctgaagtgttccttccatgttttacggcgagatggtttctcct |
| | | cgcctggccactcagccttagttgtctctgttgtcttatagaggtctacttgaagaaggaaaaacagggg |
| | | gcatggtttgactgtcctgtgagcccttcttccctgcctcccccactcacagtgacccggaatccctcga |
| | | catggcagtctagcactagtgcggccgcagatctgcttcctcgctcactgactcgctgcgctcggtcgt |
| | | tcggctgcggcgagcggtatcagctcactcaaaggcggtaatacggttatccacagaatcaggggat |
| | | aacgcaggaaagaacatgtgagcaaaaggccagcaaaaggccaggaaccgtaaaaaggccgcgtt |
| | | gctggcgtttttccataggctccgcccccctgacgagcatcacaaaaatcgacgctcaagtcagaggt |
| | | ggcgaaacccgacaggactataaagataccaggcgtttccccctggaagctccctcgtgcgctctcct |
| | | gttccgaccctgccgcttaccggatacctgtccgcctttctcccttcgggaagcgtggcgctttctcata |
| | | gctcacgctgtaggtatctcagttcggtgtaggtcgttcgctccaagctgggctgtgtgcacgaacccc |
| | | ccgttcagcccgaccgctgcgccttatccggtaactatcgtcttgagtccaacccggtaagacacgact |
| | | tatcgccactggcagcagccactggtaacaggattagcagagcgaggtatgtaggcggtgctacaga |
| | | gttcttgaagtggtggcctaactacggctacactagaagaacagtatttggtatctgcgctctgctgaag |
| | | ccagttaccttcggaaaaagagttggtagctcttgatccggcaaacaaaccaccgctggtagcggtgg |
| | | tttttttgtttgcaagcagcagattacgcgcagaaaaaaaggatctcaagaagatcctttgatcttttctac |
| | | ggggtctgacgctcagtggaacgaaaactcacgttaagggattttggtcatgagattatcaaaaaggat |
| | | cttcacctagatccttttaaattaaaaatgaagttttaaatcaatctaaagtatatatgagtaaacttggtctg |
| | | acagttaccaatgcttaatcagtgaggcacctatctcagcgatctgtctatttcgttcatccatagttgcct |
| | | gactccccgtcgtgtagataactacgatacgggagggcttaccatctggccccagtgctgcaatgata |
| | | ccgcgagacccacgctcaccggctccagatttatcagcaataaaccagccagccggaagggccgag |
| | | cgcagaagtggtcctgcaactttatccgcctccatccagtctattaattgttgccgggaagctagagtaa |
| | | gtagttcgccagttaatagtttgcgcaacgttgttgccattgctacaggcatcgtggtgtcacgctcgtcg |
| | | tttggtatggcttcattcagctccggttcccaacgatcaaggcgagttacatgatcccccatgttgtgcaa |
| | | aaaagcggttagctccttcggtcctccgatcgttgtcagaagtaagttggccgcagtgttatcactcatg |
| | | gttatggcagcactgcataattctcttactgtcatgccatccgtaagatgcttttctgtgactggtgagtact |
| | | caaccaagtcattctgagaatagtgtatgcggcgaccgagttgctcttgcccggcgtcaatacgggata |
| | | ataccgcgccacatagcagaactttaaaagtgctcatcattggaaaacgttcttcggggcgaaaactct |
| | | caaggatcttaccgctgttgagatccagttcgatgtaacccactcgtgcacccaactgatcttcagcatct |
| | | tttactttcaccagcgtttctgggtgagcaaaaacaggaaggcaaaatgccgcaaaaaagggaataag |
| | | ggcgacacggaaatgttgaatactcatactcttcctttttcaatattattgaagcatttatcagggttattgtc |
| | | tcatgagcggatacatatttgaatgtatttagaaaaataaacaaataggggttccgcgcacatttccccg |
| | | aaaagtgccacctgacgt |

### Example 1

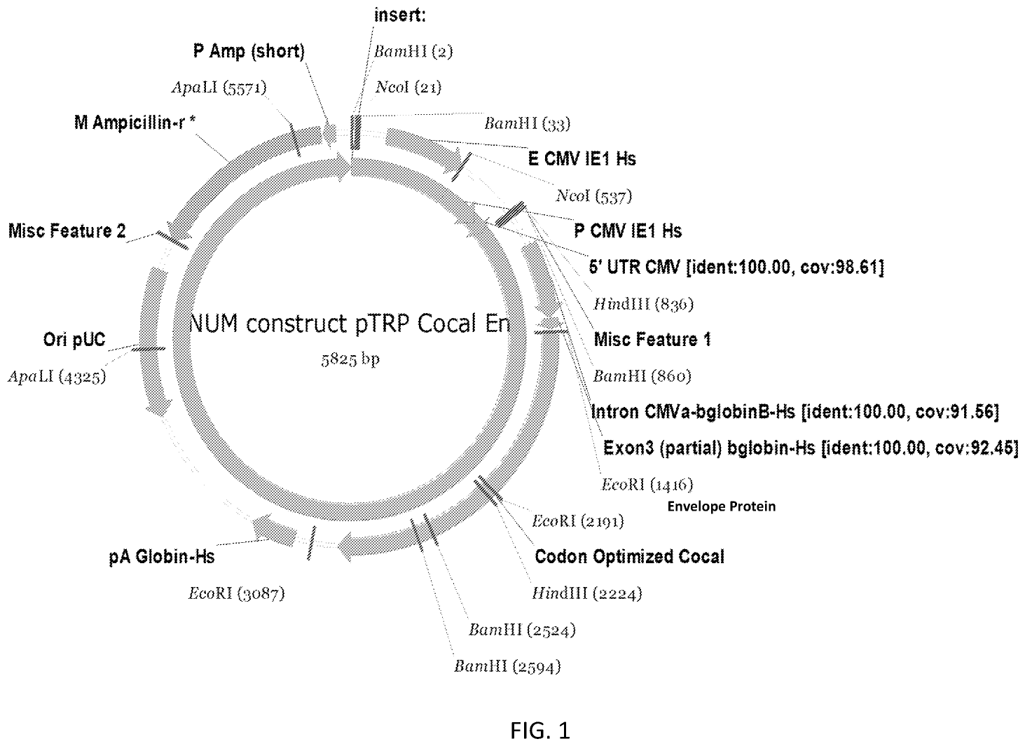

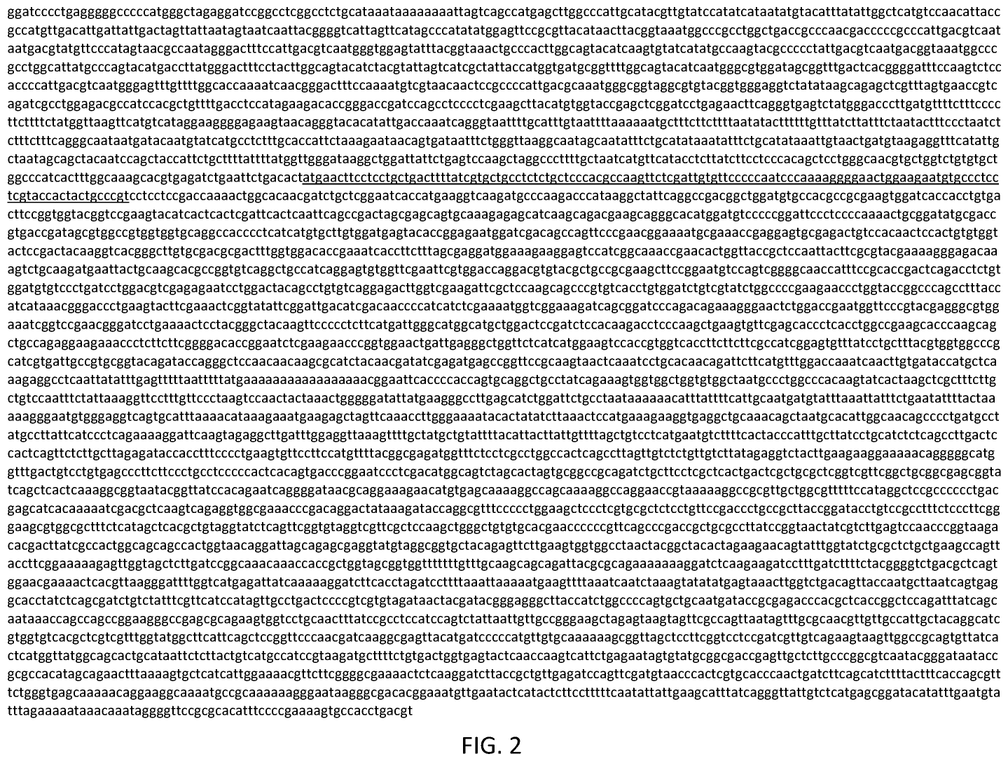

A nucleic acid sequence encoding a Cocal vesiculovirus envelope glycoprotein (Cocal-G) was codon optimized (SEQ ID NO: 1) and cloned into a pTRP expression plasmid (FIGS. 1-2) (SEQ ID NO: 4).

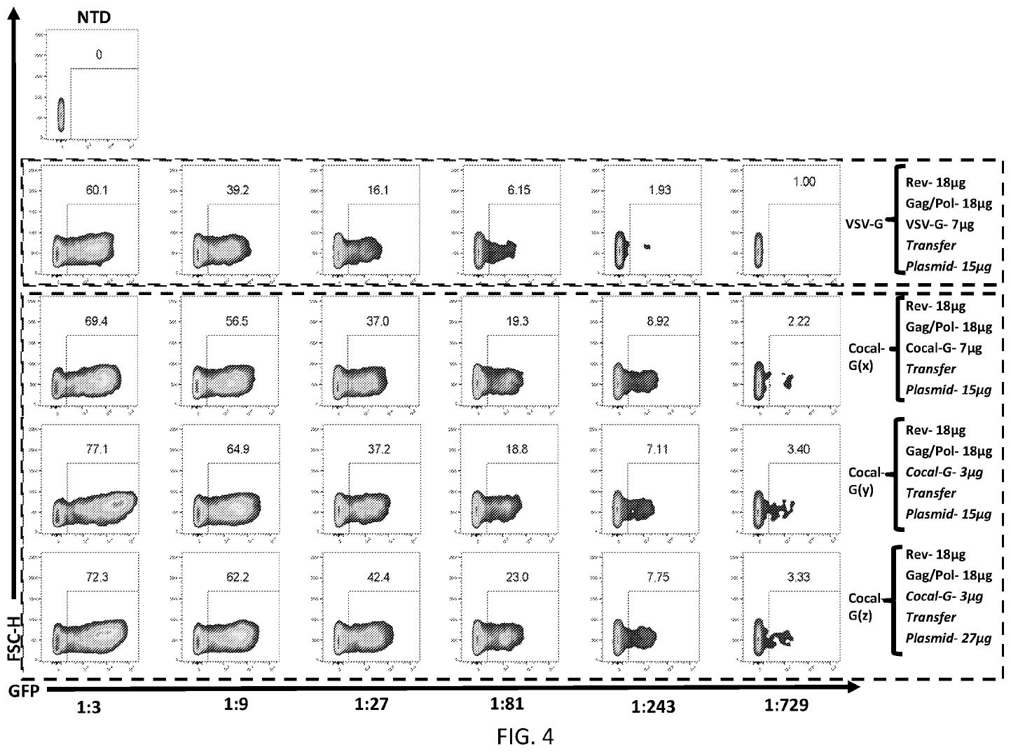

The plasmid encoding Cocal-G was transfected into HEK293-T cells along with a transfer plasmid and helper plasmids encoding Gag and Pol proteins (e.g. “gag/pol plasmid”) and the Rev protein (e.g. “rev plasmid”) (FIG. 4) to generate Cocal-G enveloped lentiviral particles. VSV-G enveloped particles were generated from HEK293-T cells transfected with a plasmid encoding the Indiana vesiculovirus (also known as vesicular stomatitis virus or vesicular stomatitis Indiana virus) envelope glycoprotein (VSV-G) along with the transfer and helper plasmids, and were used as a control.

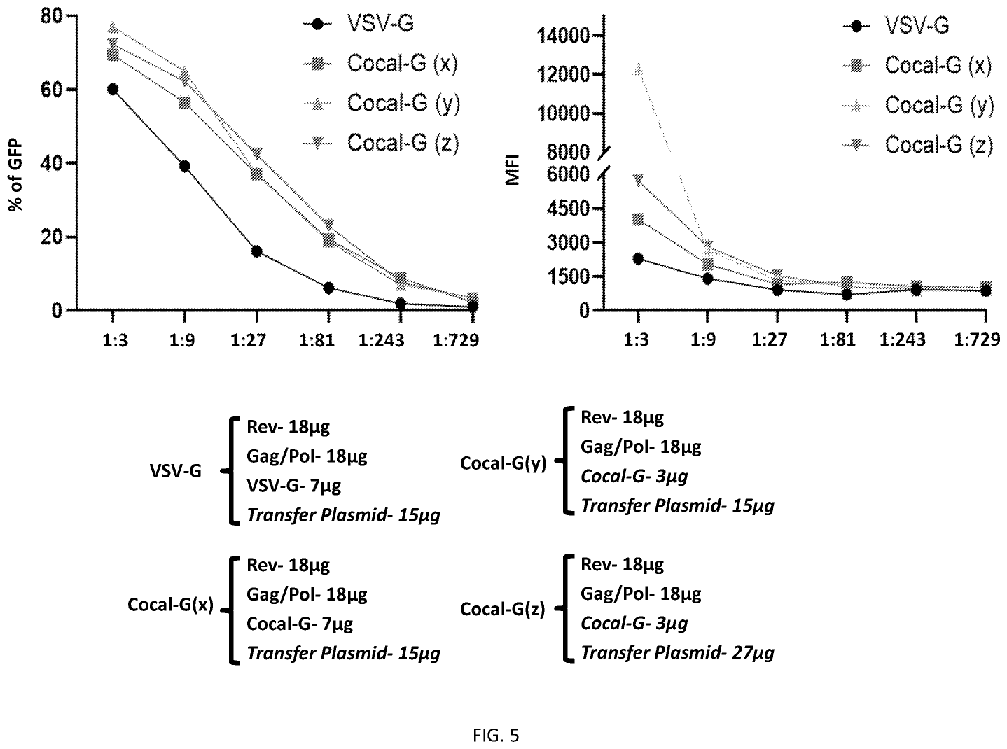

Initially, 18 μg of rev plasmid, 18 μg of gag/pol plasmid, 7 μg of VSV-G or Cocal-G plasmid, and 15 μg of transfer plasmid (i.e. the plasmid encapsulated into the retroviral particle) encoding Green fluorescent protein (GFP) was used. Cocal-G and VSV-G enveloped lentiviral particles were obtained, concentrated, and used to infect CD3/28 bead stimulated primary human CD4 T cells. Primary human CD4 T cells that were infected with the virus expressed GFP, which was detected by flow cytometry. Results showed that primary CD4 cells infected with Cocal-G enveloped lentiviral particles exhibited a higher transduction efficiency compared with cells infected with VSV-G enveloped lentiviral particles (FIG. 4, top two panels and FIG. 5).

Next, the concentration of Cocal-G plasmid was decreased (7 μg to 3 μg) and the concentration of transfer plasmid was increased (15 μg to 27 μg) (FIG. 4, bottom two panels and FIG. 5). HEK293-T cell transfection was conducted as above except for the modified concentration of the Cocal-G and transfer plasmids (FIG. 4, bottom two panels and FIG. 5). Results demonstrated that reducing the concentration of Cocal-G plasmid enhanced viral transduction efficiency (FIG. 4, bottom two panels and FIG. 5). Moreover, concomitantly increasing the concentration of the transfer plasmid while maintaining a low concentration of the Cocal-G plasmid did not impact the transduction efficiencies. Importantly, decreasing the concentration of Cocal-G plasmid results in lower cell toxicity, and increasing the concentration of transfer plasmid allows for higher titers of virus to be grown. Thus, adjusting these plasmid concentrations is crucial for scaling-up growth of lentiviral vectors and has important implications in generating safe, GMP-compliant products used for treating pateints (e.g. CAR T cells).

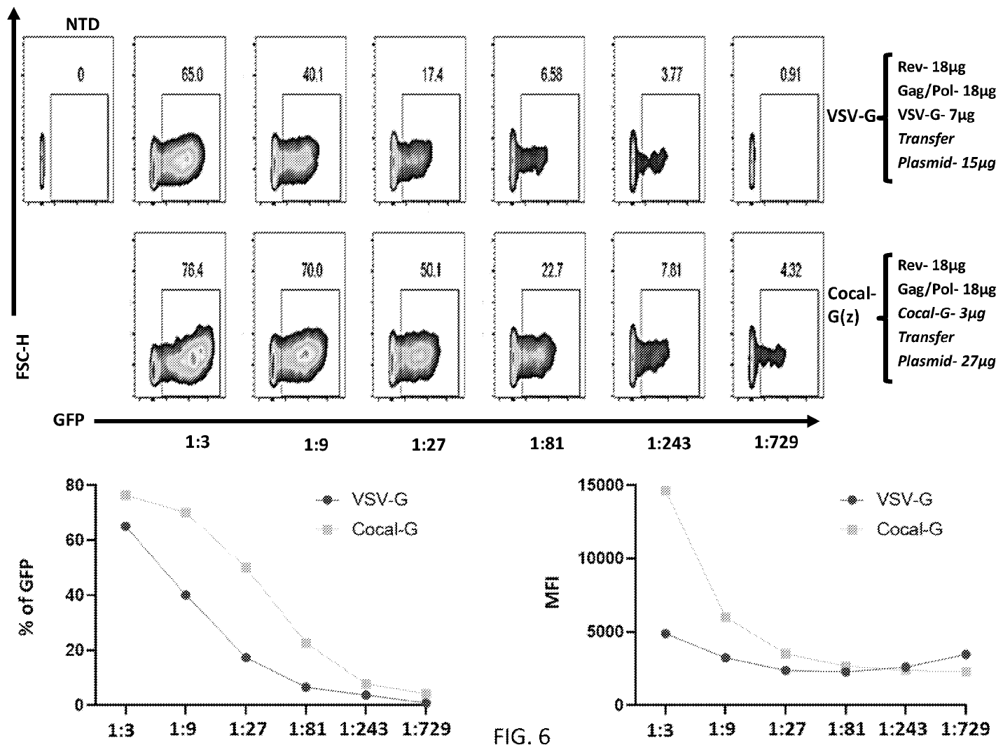

Similar results were obtained using CD8+ T cells. Cocal-G ENV lentiviral vectors generated using increased envelope (Cocal-G) plasmid and decreased transfer plasmid, enhanced transduction efficiency in CD8+ T cells (FIG. 6).

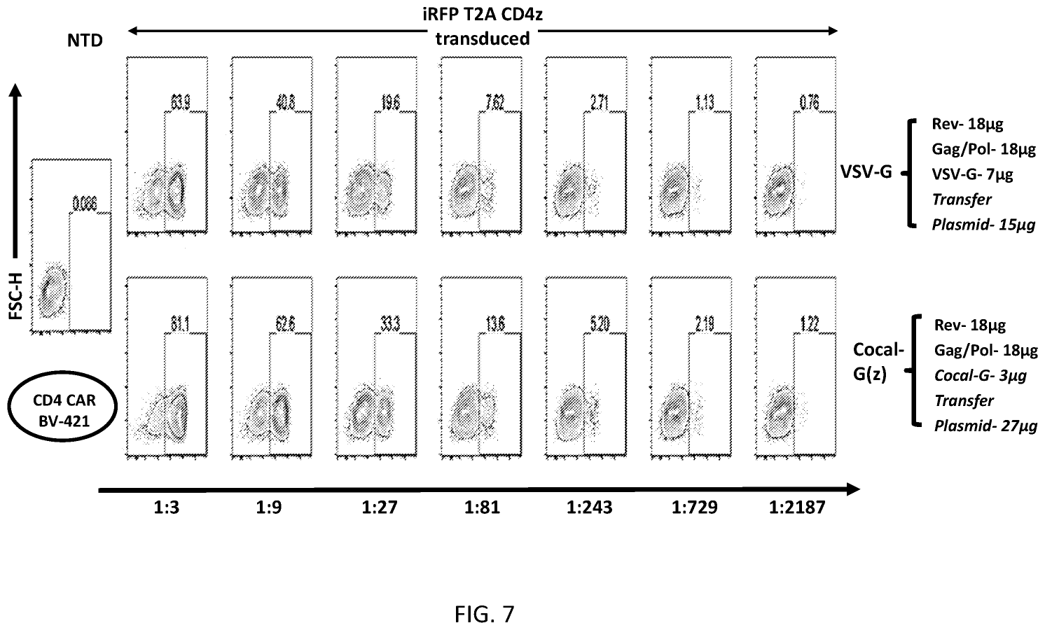

Lentiviral vectors encoding a CD4-based CAR (HIV) were generated using either Cocal or VSV-g Env. Dilutions of each vector were transduced into CD8 T cells, and cultures were stained a few days later with CD4. The data showed that vectors made with Cocal Env had a higher titer than those produced with VSV-g (FIG. 7).

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for delivering a nucleic acid encoding a chimeric antigen receptor (CAR) to an immune cell or precursor cell thereof, the method comprising introducing into the cell:
a) a transfer plasmid comprising a nucleotide sequence encoding a CAR,
b) a retroviral vector comprising a nucleotide sequence encoding a Cocal vesiculovirus envelope protein,
c) a plasmid comprising a nucleotide sequence encoding a retroviral Rev protein, and
d) at least one plasmid comprising a nucleotide sequence encoding a retroviral Gag protein and a retroviral Pol protein,
wherein the amount of transfer plasmid introduced is higher than the amount of the retroviral vector comprising a nucleotide sequence encoding a Cocal vesiculovirus envelope protein.

2. The method of claim 1, wherein the amount of transfer plasmid introduced is at least 2 times (×), 3×, 4×, 5×, 6×, 7×, 8×, 9×, 10×, or 20× the amount of the vector comprising a nucleotide sequence encoding a Cocal vesiculovirus envelope protein.

3. The method of claim 1, wherein the nucleotide sequence encoding the Cocal vesiculovirus envelope is at least 80%, at least 85%, at least 90%, at least 95%, at least 96%, at least 97%, at least 98%, at least 99%, or 100% identical to SEQ ID NO: 1.

4. The method of claim 1, wherein the expression of the envelope protein is under control of a transcriptional regulatory element.

5. The method of claim 4, wherein the transcriptional regulatory element is a eukaryotic promoter.

6. The method of claim 4, wherein the transcriptional regulatory element is a constitutive promoter.

7. The method of claim 1, wherein the Cocal vesiculovirus envelope protein comprises an amino acid sequence at least 80%, at least 85%, at least 90%, at least 95%, at least 96%, at least 97%, at least 98%, at least 99%, or 100% identical to SEQ ID NO: 2.

8. The method of claim 1, wherein the retroviral vector comprises a nucleotide sequence at least 80%, at least 85%, at least 90%, at least 95%, at least 96%, at least 97%, at least 98%, at least 99%, or 100% identical to SEQ ID NO: 4.

9. The method of claim 1, wherein the CAR comprises an antigen-binding domain, a transmembrane domain, and an intracellular domain.

10. The method of claim 9, wherein the antigen-binding domain is selected from the group consisting of a full-length antibody or antigen-binding fragment thereof, a Fab, a single-chain variable fragment (scFv), or a single-domain antibody.

11. The method of claim 9, wherein the antigen-binding domain specifically binds a target antigen selected from the group consisting of CD4, CD19, CD20, CD22, BCMA, CD123, CD133, EGFR, EGFRvIII, mesothelin, Her2, PSMA, CEA, GD2, IL-13Ra2, glypican-3, CIAX, LI-CAM, CA 125, CTAG1B, Mucin 1 (MUC1), TnMUC1, glypican-2 (GPC2), cancer cell-associated GPC2, Glycosyl-phosphatidylinositol (GPI)-linked GDNF family α-receptor 4 (GFRα4;
GFRalpha4), and Folate receptor-alpha.

12. The method of claim 9, wherein the CAR further comprises a hinge region.

13. The method of claim 9, wherein the transmembrane domain is selected from the group consisting of an artificial hydrophobic sequence, a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), ICOS (CD278), or CD154, and a transmembrane domain derived from a killer immunoglobulin-like receptor (KIR).

14. The method of claim 9, wherein the intracellular domain comprises a costimulatory signaling domain and an intracellular signaling domain.

15. The method of claim 14, wherein the intracellular domain comprises a costimulatory domain of a protein selected from the group consisting of a TNFR superfamily protein, CD27, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR), or a variant thereof.

16. The method of claim 14, wherein the intracellular signaling domain comprises an intracellular domain selected from the group consisting of cytoplasmic signaling domains of a human CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

17. The method of claim 1, wherein the immune cell is a T cell, a natural killer cell, a cytotoxic T lymphocyte, or a regulatory T cell.

18. The method of claim 17, wherein the T cell is a CD8+ T cell.

19. The method of claim 18, wherein the T cell is a CD4+ T cell.

20. The method of claim 17, wherein the T cell is a regulatory T cell.

21. The method of claim 1, wherein the retroviral vector is selected from the group consisting of a lentiviral vector, an alpharetroviral, a betaretroviral, a gammaretroviral, a deltaretrovirus, and an epsilonretrovirus.

22. The method of claim 1, wherein the Cocal vesiculovirus envelope protein is human codon-optimized.

23. The method of claim 1, wherein the method is scaled-up.

24. The method of claim 1, further comprising adapting the cells for growth in suspension.

25. The method of claim 1, further comprising adapting the cells to grow in serum-free cultures.

26. A composition comprising an immune cell or precursor cell thereof comprising a CAR, wherein the cell is produced by the method of claim 1.

27. The composition of claim 26, wherein the composition is GMP compliant.

28. A method for delivering a nucleic acid sequence encoding a chimeric antigen receptor (CAR) to an immune cell or precursor cell thereof, the method comprising transducing the cell with a Cocal vesiculovirus envelope pseudotyped retroviral particle generated in a host cell, wherein the Cocal vesiculovirus envelope pseudotyped retroviral particle comprises:
a transfer plasmid comprising a nucleotide sequence encoding a CAR,
a retroviral vector comprising a nucleotide sequence encoding a Cocal vesiculovirus envelope protein,
a plasmid comprising a nucleotide sequence encoding a retroviral Rev protein, and
at least one plasmid comprising a nucleotide sequence encoding a retroviral Gag protein and a retroviral Pol protein.

29. A method for delivering a nucleic acid sequence encoding a chimeric antigen receptor (CAR) to an immune cell, the method comprising:
a) introducing into a host cell
a transfer plasmid comprising a nucleotide sequence encoding a CAR,
a retroviral vector comprising a nucleotide sequence encoding a Cocal vesiculovirus envelope protein,
a plasmid comprising a nucleotide sequence encoding a retroviral Rev protein, and
at least one plasmid comprising a nucleotide sequence encoding a retroviral Gag protein and a retroviral Pol protein,
wherein the host cell produces a Cocal vesiculovirus envelope pseudotyped retroviral particle;
b) harvesting the Cocal vesiculovirus envelope pseudotyped retroviral particle; and
c) transducing the immune cell with the Cocal vesiculovirus envelope pseudotyped retroviral vector particle, wherein the transduced immune cell expresses the CAR encoded by the nucleotide sequence of the transfer plasmid.

30. The method of claim 28, wherein the amount of transfer plasmid introduced into the host cell is higher than the amount of the retroviral vector comprising a nucleotide sequence encoding a Cocal vesiculovirus envelope protein.
